(19) United States
(12) Patent Application Publication
(10) Pub. No.: US 2021/0128840 A1
(43) Pub. Date: May 6, 2021

(54) STRETCHABLE PLUNGER ASSEMBLIES

(71) Applicant: SIO2 MEDICAL PRODUCTS, INC., Auburn, AL (US)

(72) Inventors: Brian Russell Lilly, Auburn, AL (US); Timothy M. Miller, Collegeville, PA (US); Shankara Narayanappa, Auburn, AL (US)

(21) Appl. No.: 17/046,679

(22) PCT Filed: Apr. 9, 2019
(86) PCT No.: PCT/US2019/026680

§ 371 Date: Oct. 9, 2020

Related U.S. Application Data
(60) Provisional application No. 62/654,663, filed on Apr. 9, 2018.
(60) Provisional application No. 62/666,450, filed on May 3, 2018.
(60) Provisional application No. 62/672,934, filed on May 17, 2018.

(51) Int. Cl. A61M 5/315; A61F 9/00; A61M 5/31; A61K 9/00; C07K 16/22; A61K 38/17

(52) U.S. Cl. CPC A61M 5/31511; A61F 9/0008; A61M 5/3129; A61K 9/0048; C07K 2319/30; C07K 16/22; A61K 38/179; A61M 2205/0238; C07K 2317/24; A61K 9/0019

(57) ABSTRACT

Disclosed are plunger assemblies which include a plunger sleeve, a plunger rod and an axial protrusion disposed within an inner cavity of the plunger sleeve. Application of a distal force onto the plunger via the plunger rod causes the axial protrusion to contact and apply pressure to an engagement surface in the inner cavity. The engagement surface is configured to receive distal force from the end of the axial protrusion. This causes the plunger to elongate and slightly constrict, thus reducing break loose force and facilitating transition from storage mode to dispensing mode of the plunger.

### CROSS-REFERENCE TO RELATED APPLICATIONS

The present application claims priority to U.S. Provisional Patent Application Nos. 62/654,663, filed Apr. 9, 2018, 62/666,450, filed May 3, 2018 and 62/672,934, filed May 17, 2018, all of which are herein incorporated by reference in their entireties.

### BACKGROUND OF THE INVENTION

### 1. Field of Invention

The disclosed concept relates to plungers and their use in drug delivery devices, such as (pre-filled, filled before use or empty) syringes, cartridges or auto-injectors. More particularly, the disclosed concept relates, among other things, to stretchable plungers that provide and maintain container closure integrity in a storage mode, during the shelf-life of a pre-filled syringe. These plungers are convertible to a dispensing mode by actuating the plunger so as to stretch it, which helps facilitate low and smooth plunger force when dispensing syringe contents.

### 2. Description of Related Art

The present disclosure predominantly describes use of plungers and plunger assemblies according to the disclosed concept in connection with pre-filled syringes. However, the invention is not limited to pre-filled syringes, but may include other drug delivery devices, such as (pre-filled, filled before use, or empty) syringes, cartridges and auto-injectors.

Pre-filled parenteral containers, such as syringes or cartridges, are commonly prepared and sold so that the syringe does not need to be filled by the patient or caregiver before use. The syringe, and more specifically the barrel of the syringe, may be prefilled with a variety of different injection products, including, for example, saline solution, a dye for injection, or a pharmaceutically active preparation, among other items. This is particularly the case for syringes that are used to dispense very small and precise amounts of injectable product, such as for ophthalmic use.

Pre-filled parenteral containers are typically sealed with a rubber plunger, which provides closure integrity over the shelf life of the container's contents. To use the prefilled syringe, the packaging and cap are removed, optionally a hypodermic needle or another delivery conduit is attached to the dispensing end of the barrel, the delivery conduit or syringe is moved to a use position (such as by inserting it into a patient's blood vessel or into apparatus to be rinsed with the contents of the syringe), and the plunger is advanced axially down the barrel to inject contents of the barrel to the point of application.

Seals provided by rubber plungers in the barrel typically involve the rubber of the plunger being pressed against the barrel. Typically the rubber plunger is larger in diameter than the internal diameter of the barrel. Thus, to displace the rubber plunger when the injection product is to be dispensed from the syringe requires overcoming this pressing force of the rubber plunger. Moreover, not only does this pressing force provided by the rubber seal typically need to be overcome when initially moving the plunger, but this force also needs to continue to be overcome as the rubber plunger is displaced along the barrel during the dispensing of the injection product. The need for even slightly elevated forces to advance the plunger in the syringe may increase the difficulty a user may have in dispensing the injection product from the syringe. Such elevated forces may also hinder a user's ability to dispense small and precise amounts, such as during a priming step with an ophthalmic syringe. Such elevated forces can prove particularly problematic for auto injection systems where the syringe is placed into the auto injection device and the plunger is advanced by a fixed spring. Accordingly, primary considerations concerning the use of a plunger in a pre-filled parenteral container include: (1) adequacy of the seal provided by the plunger within the container during storage and use, for example whether the plunger provides container closure integrity (“CCI”, defined below); and (2) plunger force (defined below) required to dispense syringe contents.

In practice, CCI and plunger force tend to be competing considerations. In other words, absent other factors, the tighter the fit between the plunger and the interior surface of the container to maintain adequate CCI, the greater the force necessary to advance the plunger in use. In the field of medical syringes, it is important to ensure that the plunger can move at a substantially constant speed and with a substantially constant force when advanced in the barrel. In addition, the force necessary to initiate plunger movement and then continue advancement of the plunger should be low enough to enable precise administration by a user and comfort for a patient.

Plunger force is essentially a function of the coefficients of friction of each of the contacting surfaces (i.e., the plunger surface and interior syringe wall surface) and the normal force exerted by the plunger against the interior wall of the syringe. The greater the respective coefficients of friction and the greater the normal force, the more force required to advance the plunger. Accordingly, efforts to improve plunger force should be directed to reducing friction and lowering normal force between contacting surfaces. However, such efforts should be tempered by the need to maintain an adequate seal, e.g., CCI, as discussed above.

To reduce friction and thus improve plunger force, lubrication may be applied to the plunger, the interior surface of the container, or both. Liquid or gel-like flowable lubricants, such as free silicone oil (e.g., polydimethylsiloxane or “PDMS”), may provide a desired level of lubrication to optimize plunger force. Flowable lubricants, when used with pre-filled syringes, may migrate away from the plunger over time, resulting in spots between the plunger and the interior surface of the container with little or no lubrication. This may cause a phenomenon known as “sticktion,” an industry term for the adhesion between the plunger and the barrel that needs to be overcome to break out the plunger and allow it to begin moving.

There is a need for optimizing plunger force in a parenteral container while maintaining adequate CCI to prevent drug leakage, protect the drug product and attain sufficient product shelf life. There is also a need to achieve these ends with a plunger that cannot be pulled backwards while in a syringe barrel, e.g., for ophthalmic use.

### SUMMARY OF THE INVENTION

Accordingly, in one optional embodiment, a plunger assembly for use in a medical barrel is provided. The plunger assembly includes a plunger rod, an axial protrusion and a plunger. The plunger rod has a distal end and a proximal end. The axial protrusion is secured to, extends from or abuts the distal end of the plunger rod. The plunger includes a plunger sleeve having an exterior surface and an interior surface surrounding an inner cavity. The exterior surface includes a distal nose cone and an outer annular wall extending proximally from the nose cone and leading to an opening at a proximal end of the plunger sleeve. The opening receives the axial protrusion such that the axial protrusion extends into the inner cavity and contacts an engagement surface of the interior surface. The engagement surface is configured to receive a force applied in a distal direction by the axial protrusion to move the plunger assembly in a distal direction when the plunger rod is moved in a distal direction. The distal end of the plunger rod does not initially contact the proximal end of the plunger sleeve when the plunger is in a pre-elongation state. Application of axial force in a proximal direction onto the proximal end of the plunger rod sufficient to axially displace the proximal end of the plunger rod a predetermined distance does not axially displace the plunger in a proximal direction.

In another optional aspect, the disclosed concept relates to a plunger rod and axial protrusion provided as a single piece, of unitary construction. Alternatively, the plunger rod and axial protrusion is provided as a multi-piece assembly, wherein a first portion of the multi-piece assembly may be manually pulled apart, at least to a predetermined distance, from a second portion of the assembly.

In another optional embodiment, the disclosed concept is a prefilled syringe with the plunger of the aforementioned plunger assembly disposed within a medical barrel containing an injectable product. The plunger is configured to provide sufficient CCI and gas-tight sealing over a desired shelf life when the plunger is in storage mode. The plunger is converted to dispensing mode by axially elongating the plunger, which slightly constricts the outer annular wall of the plunger to reduce the plunger's radial compression against the barrel inner wall. This renders it easier to advance the plunger down the barrel, while still maintaining at least a liquid tight seal.

Optionally, in any embodiment, the axial protrusion and/or the interior surface of the plunger comprises a flowable lubricant, such as silicone oil. Optionally, in any embodiment, the axial protrusion and/or the interior surface of the plunger comprise a lubricity coating, optionally wherein the lubricity coating is a coating applied using plasma enhanced chemical vapor deposition (“PECVD”) having one of the following atomic ratios: SiwOxCy or SiwNxCy, where w is 1, x is from about 0.5 to 2.4 and y is from about 0.6 to about 3. Such lubrication may help facilitate movement of the axial protrusion out of the plunger, if desired.

Optionally, in any embodiment, the plunger is made from a thermoplastic elastomer or rubber, optionally a bromobutyl rubber, optionally having a durometer of from 30 to 70, preferably from 40 to 60. Optionally, in any embodiment, the outer annular wall of the plunger comprises at least one annular rib, optionally at least two annular ribs, optionally at least three annular ribs.

Optionally, in any embodiment, the disclosed concept relates to a syringe comprising a medical barrel with a plunger disposed therein, the plunger being a component of any embodiment of a plunger assembly described herein. Such a syringe is optionally a pre-filled syringe comprising an injectable product stored within a product containing area. In any embodiment, the plunger comprises a stretch zone adapted to undergo elongation along a central axis of the plunger upon application of a force in the distal direction by the axial protrusion onto the engagement surface of the inner cavity of the plunger. Such elongation reduces an outer profile of the outer annular wall along the stretch zone. Optionally, the elongation of the plunger is less than 1.5 mm.

In any syringe embodiment, the plunger rod does not initially contact the plunger sleeve when the plunger is in the pre-elongation state. Once the plunger is transitioned to dispensing mode, wherein the plunger undergoes elongation and displacement down the barrel, in some embodiments the plunger does not contact the plunger sleeve while in other embodiments it does.

In any embodiment, elongation of the plunger constricts the outer annular wall along the stretch zone, thereby reducing radial compression of the outer annular wall against the inner wall of the medical barrel.

Optionally, in any embodiment, the engagement surface is provided on a distal section of the interior surface of the inner cavity of the plunger and a distal portion of the axial protrusion, optionally solely the distal portion of the axial protrusion, contacts the engagement section.

Optionally, in any embodiment, the plunger is configured to be translated solely in a distal direction by the plunger rod.

Optionally, in any embodiment of a pre-filled syringe, when in storage mode, the plunger exerts outward radial compression against the inner wall of the medical barrel to form a liquid tight, CCI and gas-tight interface therewith. After the plunger is converted to dispensing mode, it continues to maintain a liquid tight interface and optionally maintains a CCI and gas-tight interface as the plunger is advanced down the barrel to dispense an injectable product.

Optionally, in any embodiment of a pre-filled syringe, flowable lubricant, such as silicone oil, is coated onto the syringe sidewall and/or the outer annular wall of the plunger. Optionally, in an alternative embodiment, no flowable lubricant is provided between the plunger and the syringe sidewall.

Optionally, in any embodiment of a pre-filled syringe, break loose force of the plunger is below 10 N, optionally below 9 N, optionally below 8 N, optionally below 7 N, optionally below 6 N, optionally from 4 to 8 N, optionally from 4 to 6 N. Optionally, this break loose force is achieved without a flowable lubricant between the plunger and the syringe sidewall. Optionally, in any embodiment of a prefilled syringe, the differential between break loose force and glide force is below 6 N, optionally below 4 N, optionally below 3 N, optionally below 2 N, optionally below 1.5 N, optionally below 1.0 N, optionally below 0.5 N, optionally below 0.25 N, optionally from 0.5 N to 4 N.

Optionally, in any embodiment, the plunger comprises a fluoropolymer film coating applied on its outer surface. This may provide a drug contacting surface and may optionally extend along at least a portion of the outer annular wall of the plunger so as to provide lubricity to the plunger to reduce plunger force.

### BRIEF DESCRIPTION OF SEVERAL VIEWS OF THE DRAWINGS

The invention will be described in conjunction with the following drawings in which like reference numerals designate like elements and wherein:

FIG. 1 is an isometric view of an exemplary pre-filled syringe assembly with which the disclosed concept may be implemented.

FIG. 2 is an isometric view of an exemplary one-piece plunger rod and axial protrusion assembly in accordance with a first optional embodiment of the disclosed concept.

FIG. 3A is an isometric view of an exemplary multi-piece plunger rod and axial protrusion assembly in accordance with a second optional embodiment of the disclosed concept, shown here in a collapsed position configured for downwardly advancing a syringe plunger within the syringe barrel.

FIG. 3B is an isometric view of the multi-piece plunger rod of FIG. 3B, shown here in a partially extended position.

FIG. 4 is an isolated axial sectional view of an exemplary plunger that may be used according to any embodiment of the disclosed concept.

FIG. 5 is an axial sectional view of a partial syringe assembly comprising the one-piece plunger rod of FIG. 2 with an axial protrusion inserted into a plunger within the syringe barrel.

FIG. 5A is an enlarged sectional view of a first alternative embodiment of the inner surface of the syringe of FIG. 5, comprising a tri-layer coating set disposed thereon.

FIG. 5B is an enlarged section view of a second alternative embodiment of the inner surface of the syringe of FIG. 5, comprising a four layer coating set disposed thereon.

FIG. 5C is an enlarged sectional view of a third alternative embodiment of the inner surface of the syringe of FIG. 5, comprising an organo-siloxane coating disposed thereon.

FIG. 6 is an axial sectional view of the partial syringe assembly of FIG. 5, wherein the axial protrusion and plunger rod are withdrawn from the plunger.

FIG. 7 is an axial sectional view of a partial syringe assembly comprising the multi-piece plunger rod and axial protrusion assembly of FIGS. 3A and 3B, shown in a partially extended position.

FIG. 8 is an axial sectional view of the partial syringe assembly of FIG. 7 with the multi-piece plunger rod and axial protrusion assembly shown in a collapsed position.

FIG. 9 is an axial sectional view of a partial syringe assembly comprising an exemplary two-piece plunger rod and axial protrusion assembly, in accordance with a third optional embodiment of the disclosed concept, shown here in an extended position in which the rod is separated from the rod extension disposed within the plunger.

FIG. 10 is an axial sectional view of the partial syringe assembly of FIG. 9 with the two-piece plunger rod and axial protrusion shown in an assembled position wherein a distal end of the rod abuts a proximal end of the rod extension disposed within the plunger.

FIGS. 11A-11C are partial axial sectional views of the partial syringe assembly of FIG. 5, illustrating stretching of the plunger to transition from storage mode to dispensing mode.

FIGS. 12A-12C are partial axial sectional views of the partial syringe assembly of FIG. 8, illustrating stretching of the plunger to transition from storage mode to dispensing mode.

FIGS. 13A-13C are enlarged sectional views of alternative embodiments of plunger assemblies according to optional aspects of the disclosed concept.

FIG. 14 is a graph showing data concerning the effect of the presence and length of the axial protrusion extending from a plunger rod on break loose force (Fᵢ).

FIG. 15 is a graph showing data concerning the effect of the presence and length of the axial protrusion of a plunger rod on break loose force (Fᵢ) after aging for specific time intervals.

### DETAILED DESCRIPTION OF PREFERRED EMBODIMENTS OF THE INVENTION

The disclosed concept will now be described more fully with reference to the accompanying drawings, in which several embodiments are shown. This invention may, however, be embodied in many different forms and should not be construed as limited to the embodiments set forth here. Rather, these embodiments are examples of the invention, which has the full scope indicated by the language of the claims. Like numbers refer to like elements throughout. Unless indicated otherwise, the features characterizing the embodiments and aspects described in the following may be combined with each other, and the resulting combinations are also embodiments of the present invention.

### Definitions

As used in this disclosure, an “organosilicon precursor” is a compound having at least one of the linkages:

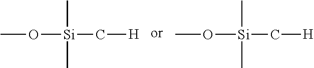

which is a tetravalent silicon atom connected to an oxygen or nitrogen atom and an organic carbon atom (an organic carbon atom being a carbon atom bonded to at least one hydrogen atom). A volatile organosilicon precursor, defined as such a precursor that can be supplied as a vapor in a plasma enhanced chemical vapor deposition (PECVD) apparatus, is an optional organosilicon precursor. Optionally, the organosilicon precursor is selected from the group consisting of a linear siloxane, a monocyclic siloxane, a polycyclic siloxane, a polysilsesquioxane, an alkyl trimethoxysilane, a linear silazane, a monocyclic silazane, a polycyclic silazane, a polysilsesquiazane, and a combination of any two or more of these precursors. Preferably, the organosilicon precursor is octamethylcyclotetrasiloxane (OMCTS). Values of w, x, y, and z are applicable to the empirical composition Si_{w}OₓC_{y}H_{z} throughout this specification. The values of w, x, y, and z used throughout this specification should be understood as ratios or an empirical formula (for example for a coating or layer), rather than as a limit on the number or type of atoms in a molecule. For example, octamethylcyclotetrasiloxane, which has the molecular composition Si₄O₄C₈H₂₄, can be described by the following empirical formula, arrived at by dividing each of w, x, y, and z in the molecular formula by 4, the largest common factor: Si₁O₁C₂H₆. The values of w, x, y, and z are also not limited to integers. For example, (acyclic) octamethyltrisiloxane, molecular composition Si₃O₂C₈H₂₄, is reducible to Si₁O_{0.67}C_{2.67}H₈. Also, although SiOₓC_{y}H_{z} is described as equivalent to SiOₓC_{y}, it is not necessary to show the presence of hydrogen in any proportion to show the presence of SiOₓC_{y}.

“Container closure integrity” or “CCI” refers to the ability of a container closure system, e.g., a plunger disposed in a prefilled syringe barrel, to provide protection and maintain efficacy and sterility during the shelf life of a sterile product contained in the container.

The “plunger sliding force” (synonym to “glide force,” “maintenance force”, or Fₘ, also used in this description) in the context of the present invention is the force required to maintain movement of a plunger tip in a syringe barrel, for example during aspiration or dispense. It can advantageously be determined using the ISO 7886-1:1993 test known in the art. A synonym for “plunger sliding force” often used in the art is “plunger force” or “pushing force.”

The “plunger breakout force” (synonym to “breakout force”, “break loose force”, “initiation force”, Fᵢ, also used in this description) in the context of the invention is the initial force required to initiate movement of the plunger in a syringe, for example in a prefilled syringe.

The term “syringe” is to be understood broadly and includes cartridges, injection “pens,” and other types of barrels or reservoirs adapted to be assembled with one or more other components to provide a functional syringe. “Syringe” also includes related articles such as auto-injectors, which provide a mechanism for dispensing the contents. Optionally, “syringe” may include prefilled syringes. A “syringe” as used herein may also apply to vaccine dispensing syringes comprising a product space containing a vaccine. A “syringe” as used herein may also have applications in diagnostics, e.g., a sampling device comprising a medical barrel prefilled with a diagnostic agent (e.g., contrast dye) or the like.

“PECVD” refers to plasma enhanced chemical vapor deposition.

### Optional Syringe Barrel Materials

Optionally, syringes according to any embodiment of the invention maybe made from one or more injection moldable thermoplastic materials including, but not limited to: an olefin polymer; polypropylene (PP); polyethylene (PE); cyclic olefin copolymer (COC); cyclic olefin polymer (COP); polymethylpentene; polyester; polyethylene terephthalate; polyethylene naphthalate; polybutylene terephthalate (PBT); PVdC (polyvinylidene chloride); polyvinyl chloride (PVC); polycarbonate; polymethylmethacrylate; polylactic acid; polylactic acid; polystyrene; hydrogenated polystyrene; poly(cyclohexylethylene) (PCHE); nylon; polyurethane polyacrylonitrile; polyacrylonitrile (PAN); an ionomeric resin; Surlyn® ionomeric resin. For applications in which clear and glass-like polymers are desired (e.g., for syringes and vials), a cyclic olefin polymer (COP), cyclic olefin copolymer (COC) or polycarbonate may be preferred. Such materials may be manufactured, e.g., by injection molding or injection stretch blow molding, to very tight and precise tolerances (generally much tighter than achievable with glass). Alternatively, syringes according to embodiments of the invention may be made from glass.

### Syringe and Plunger Assembly Components and Embodiments

As set forth above, the disclosed concept generally relates to plungers that are convertible to a dispensing mode by actuating the plunger so as to stretch it, which helps facilitate low and smooth plunger force when dispensing syringe contents. Applicant SiO2 Medical Products, Inc. has developed other convertible plungers, which are described in some of its published international patent applications, including WO2015/054282, published Apr. 16, 2015, WO2016/039816, published Mar. 17, 2016, WO2017/011599, published Jan. 19, 2017 and WO2017/209800, published Dec. 7, 2017. Each of these published applications are incorporated by reference herein in their entireties for all that they disclose.

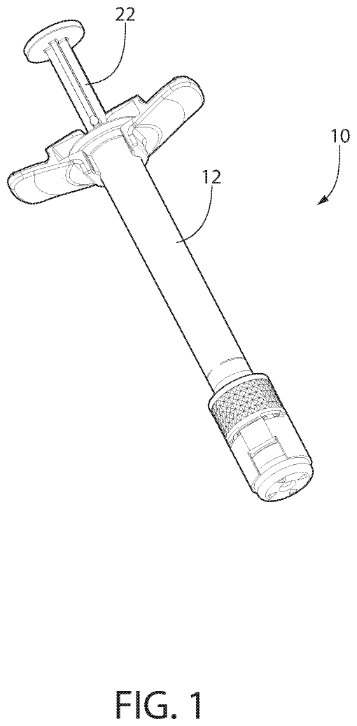

Referring to FIG. 1, there is shown an exemplary embodiment of a syringe assembly 10 (e.g., a prefilled syringe assembly) in accordance with an optional aspect of the disclosed concept. The syringe assembly includes a medical barrel 12 and a plunger assembly disposed therein, of which a portion of a plunger rod 22 is shown in FIG. 1. The syringe assembly 10 may include accoutrements typically included with prefilled syringes, such as an end cap, optionally a luer fitting to which a syringe needle may be secured at time of use, etc. Alternatively, the syringe may be a staked needle syringe.

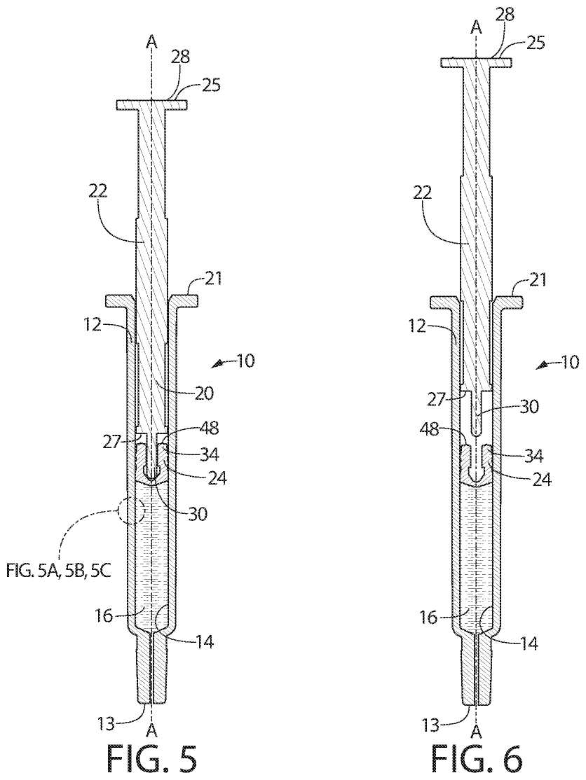

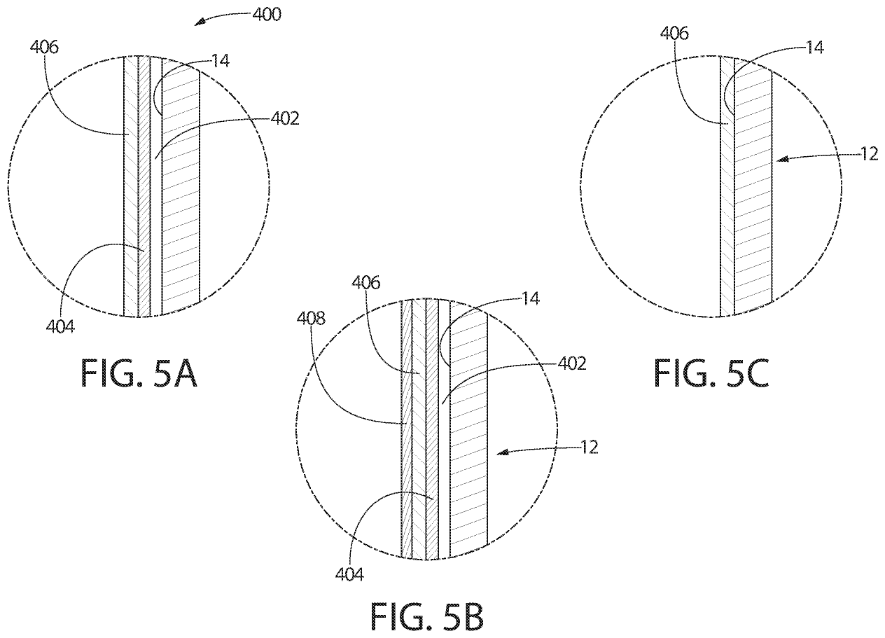

In any embodiment, for example as shown in FIGS. 5-10, the syringe assembly 10 includes a hollow medical barrel 12 having a central longitudinal axis A. The medical barrel 12 has an inner wall 14 and is configured to hold an injectable liquid 16, optionally a drug product, therein. The injectable liquid 16 is preferably prefilled so as to provide a prefilled syringe. In an alternative embodiment, the syringe is not prefilled. A needle (not shown) may be provided at the distal end of the medical barrel 12 to dispense the injectable liquid 16.

The terms “distal” and “proximal” are used throughout this specification. The terms “distal” and “proximal” refer generally to a spatial or positional relationship relative to a given reference point, wherein “proximal” is a location at or comparatively closer to that reference point and “distal” is a location further from that reference point. As applied herein to medical barrels, referring to FIG. 5, the relevant reference point is the back end of the barrel, for example, the flange 21 at the top of the barrel 12. The distal end is at the bottom or dispensing end 13 of the barrel 12, where a needle may be mounted. This same convention applies to other components described herein, such as plungers and plunger assemblies. “Proximal” and “distal” may also be used to refer to force vectors and direction of displacement. For example, the pushing force to dispense syringe contents would be applied in a “distal direction” or “distally,” i.e., a force pushing a plunger to advance it down toward the dispensing end or distal end of the medical barrel. By contrast, a pulling force on a plunger rod to pull it away from the dispensing end of the barrel would be a force applied in a “proximal direction” or “proximally.”

In any embodiment of a syringe assembly, or as aspect in and of itself of the disclosed concept, a plunger assembly 20, 120, 220 is provided and shown in FIGS. 5-10. The plunger assembly 20, 120, 220 comprises a plunger rod 22, 122, 222, an axial protrusion 30, 130, 230 secured to, extending from or abutting the distal end 27, 127, 227 of the plunger rod 22, 122, 222 and a plunger 24 into which the axial protrusion 30, 130, 230 is disposed. These assemblies are discussed in greater detail below.

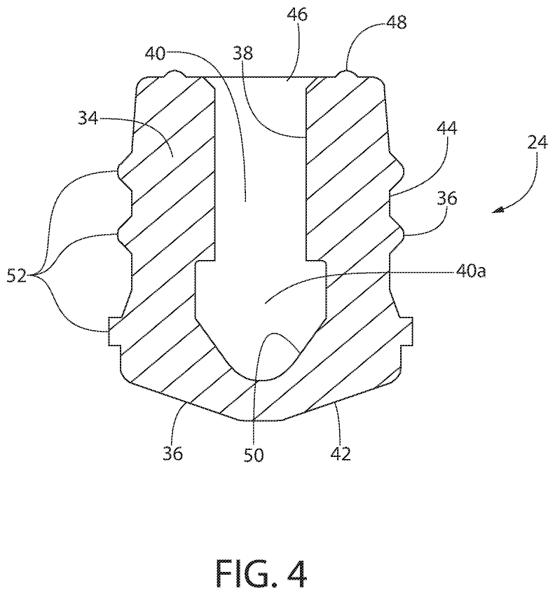

An exemplary plunger 24 usable in accordance with aspects of the disclosed concept is shown in FIG. 4. The plunger 24 comprises a plunger sleeve 34 having an exterior surface 36 and an interior surface 38 surrounding an inner cavity 40. The exterior surface 36 comprises a distal nose cone 42 and an outer annular wall 44 extending proximally therefrom. The outer annular wall 44 may include one or more ribs 52 and leads to an opening 46 at a proximal end 48 of the plunger sleeve 34. The opening 46 is configured to receive the axial protrusion 30, 130, 230 as discussed above, such that the axial protrusion 30, 130, 230 extends into the inner cavity 40 and contacts an engagement surface 50 of the interior surface 38. The engagement surface 50 is configured to receive a force applied in a distal direction by the plunger rod 22, 122, 222 to move the plunger assembly 20, 120, 220 in a distal direction. The inner cavity 40 optionally comprises a distal compartment 40a having a wider internal geometry than that of the portion of the inner cavity 40 leading up to the distal compartment 40a.

When assembled with the plunger assembly 20, 120, 220 and disposed within a medical barrel 12, the plunger 24 is configured to provide sufficient compressive force against the inner wall 14 of a prefilled syringe or cartridge barrel to effectively seal and preserve the shelf-life of the contents of the barrel during storage. When the plunger 24 provides container closure integrity (CCI) and gas-tight sealing (e.g., providing a barrier to oxygen, moisture and/or optionally additional gases), adequate to effectively seal and preserve the shelf-life of the contents of the barrel during storage, the plunger (or at least a portion of its exterior surface) may alternatively be characterized as being in an “expanded state” or “storage mode.” The expanded state or storage mode may be a product of, for example, an expanded outer diameter or profile of at least a portion of the syringe barrel-contacting surface of the plunger and/or the normal force that the plunger exerts on the inner wall of the syringe barrel in which it is disposed. The plunger 24 (or at least a portion of its exterior surface) is reducible to what may alternatively be characterized as a “constricted state” or a “dispensing mode,” wherein the compressive force against the sidewall of the barrel is reduced or eliminated in part, allowing a user to more easily advance the plunger in the barrel and thus dispense the contents of the syringe or cartridge. As discussed in greater detail below, conversion from storage mode to dispensing mode is effectuated by elongation of the plunger 24. Prior to elongation, the plunger 24 may be said to be in its natural state or “pre-elongation state.” When the plunger is disposed within a medical barrel, the pre-elongation state is synonymous with the expanded state or storage mode.

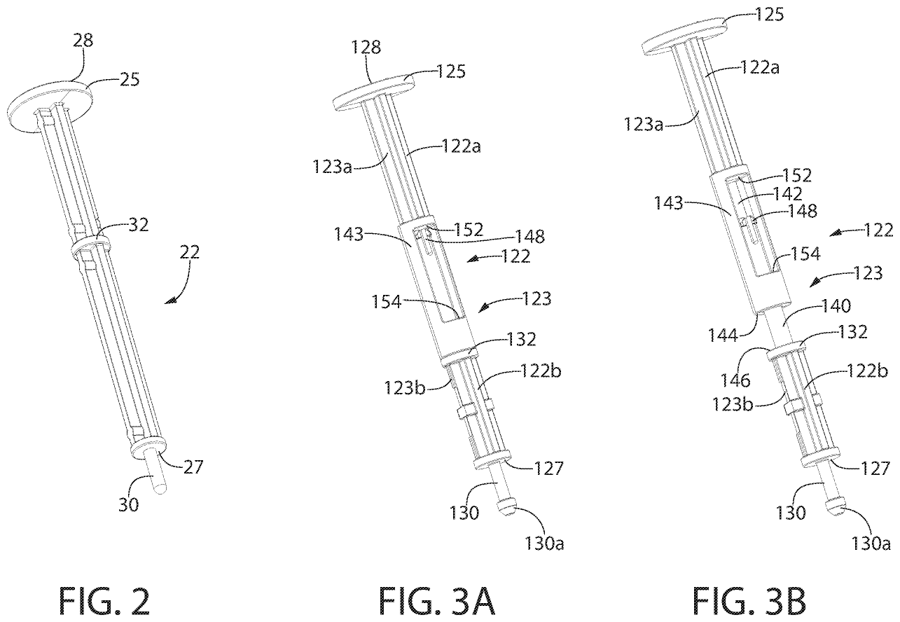

In accordance with the disclosed concept, the plunger rod and axial protrusion may optionally be provided as a single piece, of unitary construction, e.g., as shown in FIGS. 2, 5 and 6. In accordance with an alternative aspect of the disclosed concept, e.g., as shown in FIGS. 3A, 3B, and 7-10, the plunger rod and axial protrusion may be provided as a multi-piece assembly 123, 223 wherein a first portion 123a, 223a of the multi-piece assembly 123, 223 may be manually pulled apart, at least to a predetermined distance, from a second portion of the assembly 123b, 223b. The multi-piece assemblies 123, 223 shown are two-piece assemblies, but it should be understood that assemblies with more than two pieces may be within the scope of the disclosed concept. The aforementioned alternative embodiments are expounded upon below.

FIGS. 2, 5 and 6 show an embodiment in which the plunger rod 22 and axial protrusion 30 are of unitary construction. “Unitary construction” can imply a single manufactured piece or an assembly in which the assembled components (e.g., plunger rod and protrusion) are rigidly secured to each other so as to move together in any direction as a unitary part. The plunger rod 22 is an elongate member having a proximal end 25 and distal end 27. The plunger rod 22 comprises an optionally disc shaped thumb rest 28 at the proximal end 25. The axial protrusion 30 is secured to (and in this case, integral with) and extends from the distal end 27 of the plunger rod 22. As discussed below, the axial protrusion may be provided in alternative shapes. However, in this embodiment it is preferred that the axial protrusion 30 is generally cylindrical and of essentially uniform cross section along nearly its entire length, e.g., at least 90% of its entire length, for example up until the optionally rounded tip thereof. Optionally, the plunger rod 22 includes one or more radial stabilizing members 32, which may loosely engage the inner wall of a medical barrel when in use, to stabilize the plunger rod 22 (e.g, by preventing wobbling), as the plunger assembly 20 is advanced down the barrel.

Referring to FIGS. 3A, 3B, 7 and 8, there is shown an embodiment in which the plunger rod 122 and axial protrusion 130 are provided as a multi-piece assembly 123. A first portion 123a thereof, in this embodiment, includes a proximal plunger rod piece 122a. A second portion 123b of the assembly 123 includes a distal plunger rod piece 122b, wherein the axial protrusion 130 is secured to (and in this case, integral with) and extends from the distal end 127 of the plunger rod 122. The axial protrusion 130 optionally comprises, at a distal end thereof, a head 130a having a greater cross-sectional width or diameter than that of the section of the axial protrusion 130 leading to the head 130a. The particular geometric shape shown of the head 130a is merely exemplary and it should be understood that the head may be embodied in other shapes, for example spherical or cylindrical. The plunger rod 122 comprises an optionally disc shaped thumb rest 128 at the proximal end 125. Optionally, the plunger rod 122 includes one or more radial stabilizing members 132, as described above with respect to the embodiment of FIG. 2.

The first portion 123a and second portion 123b of the multi-piece assembly 123 are assembled together in a telescoping arrangement. The first and second portions 123a, 123b, may be axially pulled apart to a predetermined distance and collapsed until the portions cannot be pushed together any further. In the exemplary embodiment shown, the first portion includes a hub 143 having a central hollow 142 configured to receive a proximal shaft 140 of the second portion 123b. The central hollow 142 includes an upward facing wall 154 and an opposing downward facing wall 152. The first portion 123a includes a distal abutment surface 144 and the second portion 123b includes a proximal abutment surface 146. These two abutment surfaces (144 and 146) are configured to abut each other when the first portion 123a and second portion 123b are fully collapsed together. When the portions are collapsed together in this way, application of sufficient distally directed force onto the thumb rest 128 causes the multi-piece assembly 123 to move as a unit in the distal direction. In an optional alternative embodiment (not shown), the configuration is reversed, such that the hub and hollow are part of the second portion and the shaft that is disposable therein is part of the first portion.

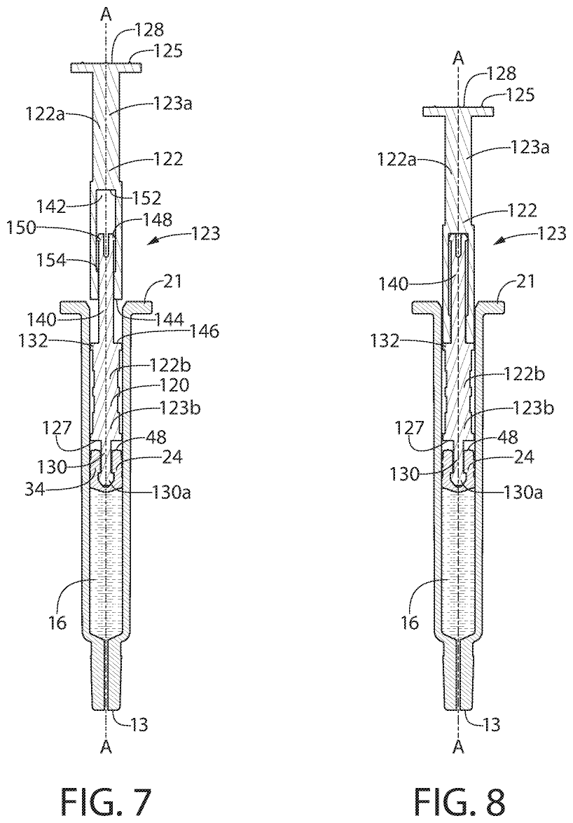

The proximal shaft 140 is movable along axis A from a fully collapsed state of the assembly 123, as shown in FIGS. 3A and 8, to a fully extended state. A partially extended state is shown in FIGS. 3B and 7. The proximal end of the proximal shaft 140 comprises prongs 148 having radial abutments 150. The radial abutments 150 are configured to abut the upward facing wall 154 when the assembly 123 is in the fully extended position, so as to prevent the first portion 123a and second portion 123b from being pulled any further apart from each other in the fully extended state. Optionally, the top ends of the prongs 148 abut the downward facing wall 152 when the assembly 123 is fully collapsed.

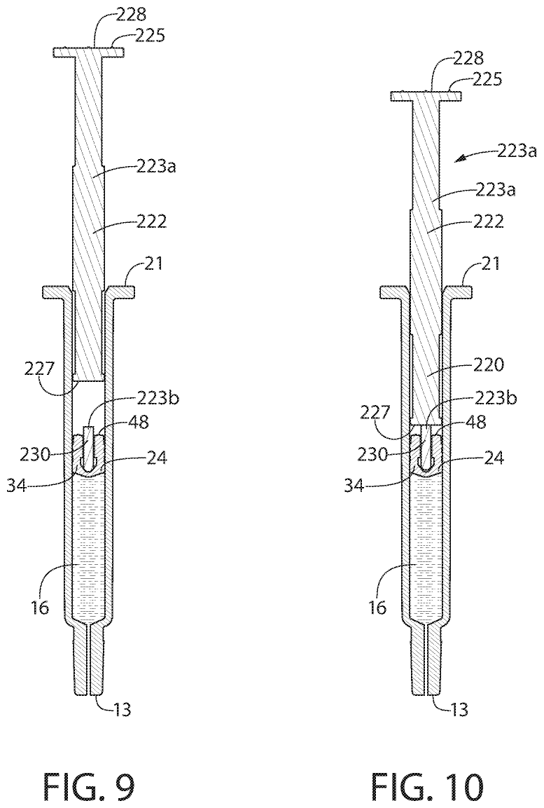

An alternative multi-piece assembly 223 is shown in FIGS. 9 and 10. The assembly includes a first portion 223a, which comprises the plunger rod 222 and a second portion 223b which comprises the axial protrusion 230. The first portion 223a and second portion 223b are not secured to each other. The distal end 227 of the plunger rod 222 is configured to abut the proximal end of the axial protrusion 230 so as to enable the plunger rod 222 to move the axial protrusion 230 in a distal direction through application of a distal force onto the plunger rod 222 (optionally via the thumb rest 228). Application of a proximal force onto the plunger rod 222 operates to completely separate the plunger rod 222, or first portion 223a of the assembly 223 from the axial protrusion 230, or second portion 223b of the assembly 223.

As discussed above, the plunger 24, as part of a plunger assembly 20, 120, 220, is configured to be disposed within a medical barrel 12 of a syringe, preferably a prefilled syringe. In that position, when sufficient distal force is applied to the plunger assembly 20, 120 220, the plunger 24 is advanced down the medical barrel 12 so as to dispense the injectable liquid 16 from the dispensing end 13 of the medical barrel 12, e.g., through a needle. When this occurs, the plunger 24 is converted from storage mode to dispensing mode. In storage mode, the plunger 24 provides a tight seal, as set forth above. This tight seal may provide a level of radial compression against the inner wall 14 of the medical barrel 12 that makes it difficult to advance the plunger down the barrel. When a user initially applies sufficient distal force onto the plunger assembly 20, 120, 220, the plunger 24 begins to stretch axially, causing at least a portion of the outer annular wall 44 of the plunger sleeve 34 to constrict slightly so as to reduce the radial compression against the inner wall 14, while still providing a liquid seal, thus providing a more desirable glide force than would be achievable without elongating the plunger sleeve 34.

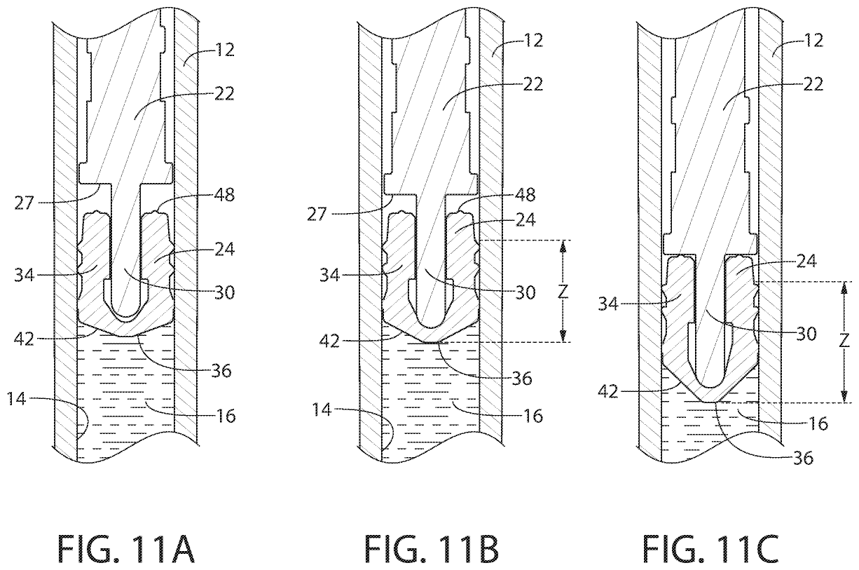

Referring now to FIGS. 11A-11C, conversion of the plunger 24 of plunger assembly 20 (FIG. 5) from storage mode to dispensing mode is illustrated. FIG. 11A shows the plunger 24 in the pre-elongation state or storage mode. In this position, the axial protrusion 30 applies little to no distal force to the engagement surface 50 of the interior surface 38 of the plunger sleeve 34. As such, the plunger is in its “fattest” state, providing its greatest radial compression against the inner wall 14 of the medical barrel 12, e.g., to provide CCI over shelf life of the product 12. The diameter or cross-sectional width of the axial protrusion 30 preferably does provide radial support to reinforce the seal that the plunger 24 provides. However, in this embodiment, it is preferred that the axial protrusion 30 is relatively loosely fitted within the inner cavity 40 of the plunger sleeve 34, without an interference fit. In this way, the axial protrusion 30 may be relatively easily withdrawn from the plunger 24 if a proximal force is applied to the plunger rod 22. Accordingly, application of axial force in a proximal direction onto the proximal end of the plunger rod 22, sufficient to axially displace the plunger rod in a proximal direction, does not axially displace the plunger 24 in a proximal direction. This would also be the case with the plunger assembly 220 of FIG. 10. The only difference is that pulling back on the plunger rod 222 of the plunger assembly 220 would completely separate the axial protrusion 230 from the plunger rod 222, such that the axial protrusion 230 remains within the plunger sleeve 34 while the plunger rod 222 is proximally displaced therefrom. By contrast, since the plunger rod 22 and axial protrusion 30 move in both axial directions as a unit, pulling back the plunger rod 22 of the plunger assembly 20 of FIG. 5 would withdraw the axial protrusion 30 from the plunger sleeve 34, as shown in FIG. 6. Optionally, a lubricant is provided within the inner cavity 40 of the plunger sleeve 34 so as to make it easier for the axial protrusion 30 to be removed therefrom when the plunger rod 22 is pulled backwards.

FIGS. 11B and 11C illustrate transition of the plunger 24 to dispensing mode. When a user applies sufficient initial distal force onto the plunger rod 22, this causes the axial protrusion 30 to apply force in a distal direction onto the engagement surface 50. Optionally, a portion of the plunger sleeve 34 may initially adhere to the inner wall via “sticktion.” As this happens, the plunger 24 axially elongates along a stretch zone Z, causing the plunger 24 to slightly constrict about the stretch zone Z. Constriction of the plunger 24 reduces radial compression onto the sidewall 14 of the medical barrel 12, thus converting the plunger 24 into dispensing mode. The plunger 24 may thus be more easily advanced down the medical barrel 12, all the while maintaining a liquid tight seal and optionally CCI.

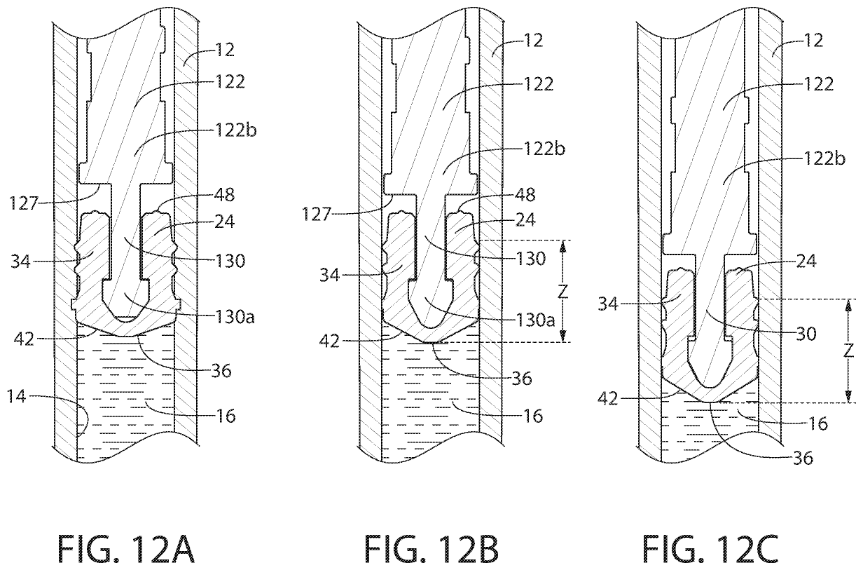

Referring now to FIGS. 12A-12C, conversion of the plunger 24 of plunger assembly 120 (FIGS. 7 and 8) from storage mode to dispensing mode is illustrated. FIG. 12A shows the plunger 24 in the pre-elongation state or storage mode. In this position, the shaft of the axial protrusion 130 applies little to no distal force to the engagement surface 50 of the interior surface 38 of the plunger sleeve 34. However, the head 130a of the axial protrusion 130, which is disposed in a distal compartment 40a within the inner cavity 40 is of a diameter or cross sectional width to abut adjacent sections of the interior surface 38. Preferably, this configuration causes a distal portion of the outer annular wall 44 of the plunger sleeve 34, optionally the rib 52 closest to the nose cone 42, to provide additional radial compression against the inner wall 14 of the medical barrel 12 (i.e., more radial compression than there would be without the head 130a).

The plunger sleeve 34 preferably includes a narrower section of the inner cavity 40 proximal to the distal compartment 40a. When the head 130a occupies the distal compartment 40a of the inner cavity 40, the axial protrusion 130 cannot be readily manually pulled out of the plunger 24 because the head 130a is of greater diameter or cross-sectional width than the narrower section of the inner cavity 40. Nevertheless, pulling back on the plunger rod 122 will not proximally displace the plunger 24. As explained above, the telescoping arrangement of the multi-piece assembly 123 is configured such that application of axial force in a proximal direction onto the proximal end of the plunger rod 122, sufficient to axially displace the proximal plunger rod piece 122a, will not also pull the axial protrusion 130 or plunger 24 in a proximal direction.

As shown in FIG. 12A, the plunger 24 is in storage mode or pre-elongation mode. In this position, the plunger 24 is in its “fattest” state, providing its greatest radial compression against the inner wall 14 of the medical barrel 12, e.g., to provide CCI over shelf life of the product 16. FIGS. 12B and 12C illustrate transition of the plunger 24 to dispensing mode. When a user applies sufficient initial distal force onto the plunger rod 122, this causes the axial protrusion 30 to apply force in a distal direction onto the engagement surface 50. Optionally, a portion of the plunger sleeve 34 may initially adhere to the inner wall via “sticktion.” As this happens, the plunger 24 axially elongates along a stretch zone Z, causing the plunger 24 to slightly constrict about the stretch zone Z. Constriction of the plunger 24 reduces radial compression onto the sidewall 14 of the medical barrel 12, thus converting the plunger 24 into dispensing mode. The plunger 24 may thus be more easily advanced down the medical barrel 12, all the while maintaining a liquid tight seal and optionally CCI.

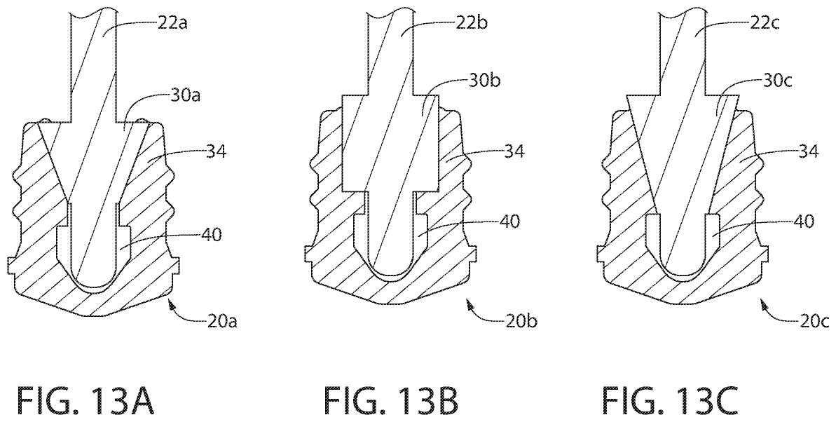

Referring to FIGS. 13A-13C, there are shown alternative embodiments of portions of plunger assemblies 20a, 20b, 20c, according to optional aspects of the disclosed concept. As with plunger assemblies 20, 120, 220, each of the plunger assemblies 20a, 20b, 20c includes a plunger 24a, 24b, 24c and a plunger rod 22a, 22b, 22c. Each plunger rod 22a, 22b, 22c has extending therefrom a uniquely shaped axial protrusion 30a, 30b, 30c. Each respective axial protrusion 30a, 30b, 30c extends into the inner cavity 40 of the plunger sleeve 34 and interfaces with portions of the interior surface of the plunger sleeve 34.

FIGS. 13A and 13C show embodiments of axial protrusions 30a, 30c that taper inward distally. In this way, it is contemplated that each axial protrusion 30a, 30c would contact the interior surface of the plunger sleeve 34 and, when pushed distally, apply force vectors thereto in both axial (distal) and radial directions. The axial force vector would cause the plunger 24a, 24c to stretch axially along a stretch zone, as explained above. The radial force vector may cause the plunger sleeve 34a, 34c to expand radially and/or to reinforce the plunger 24a, 24c from collapsing in on itself as it transitions from storage mode to dispensing mode, which may be desirable for some applications.

FIG. 13B shows an embodiment of an axial protrusion 30b, which has a proximal thicker portion. However, the axial protrusion 30b does not have any tapering sides. As such, the thicker portion reinforces the plunger 24b from collapsing in on itself. However, this configuration would only exert an axial (distal) force vector within the cavity to axially stretch the plunger, without actively expanding the plunger as the plunger is being advanced down the barrel.

Optionally, in any embodiment, the axial protrusion 30, 130, 230, is provided within the inner cavity 40 of the plunger sleeve 34 as the only component disposed therein. The axial protrusion 30, 130, 230 is not secured to the plunger 24 or to an insert within the plunger by a threaded engagement.

Optionally, in any embodiment, when the plunger 24 is in storage position, the distal end 27, 127, 227 of the plunger rod 22, 122, 222 does not contact the proximal end 48 of the plunger 24. Optionally, in some embodiments (e.g., that shown in FIGS. 7, 8 and 12A-12C and optionally in other embodiments disclosed herein), when the plunger 24 is in the dispensing position, the distal end 127 of the plunger rod 122 does not contact the proximal end 48 of the plunger 24. In other words, optionally in both storage position (pre-elongation state) and dispensing position (after elongation), a space may be provided between the proximal end 48 of the plunger 24 and the distal end of the plunger rod.

Preferably, in any embodiment, the plunger rod cannot pull the plunger backwards at any point after filling the syringe and loading the plunger. This feature is required in some applications (e.g., ophthalmic), but until development of Applicants' invention, had not been provided with a convertible plunger assembly.

Preferably, in any embodiment, the plunger cannot move backwards more than 2 mm due to an increase in pressure within the filled portion of the syringe at any point after filling the syringe and loading the plunger.

Optionally, in any embodiment in which the axial protrusion is of the same diameter along nearly its entire length (e.g., until the distal end thereof), the axial protrusion is equal to or less than 1.8 mm in diameter, optionally equal to or less than 1.6 mm in diameter. Optionally, in any such embodiment, the axial protrusion is from 1.45 mm to 1.8 mm in diameter, optionally from 1.45 mm to 1.6 mm in diameter. Optionally, where the axial protrusion 30, 230 is of the same diameter along nearly its entire length (e.g., until the distal end thereof), the diameter is such that it contacts the inner cavity 40 of the plunger sleeve 34 to reinforce the plunger's ability to provide a seal without being engaged in an interference fit with the inner cavity. As such, pulling the axial protrusion back while the plunger is in the barrel will not also pull the plunger back.

Optionally, in any embodiment, the syringe is a 0.5 mL syringe, as that term is understood in the industry.

### PECVD Coating Layers

In another aspect, the invention optionally includes use of any embodiments (or combination of embodiments) of plungers according to the disclosed concept in syringes having a PECVD coating or PECVD coating set. The syringes may be made from, e.g., glass or plastic. Optionally, the syringe barrel according to any embodiment is made from an injection moldable thermoplastic material as defined above, in particular a material that appears clear and glass-like in final form, e.g., a cyclic olefin polymer (COP), cyclic olefin copolymer (COC) or polycarbonate. Such materials may be manufactured, e.g., by injection molding, to very tight and precise tolerances (generally much tighter than achievable with glass). This is a benefit when trying to balance the competing considerations of seal tightness and low plunger force in plunger design.

This section of the disclosure focuses primarily on prefilled syringes as a preferred implementation of optional aspects of the invention. Again, however, it should be understood that the invention may include any parenteral container that utilizes a plunger, such as syringes that are empty, cartridges, auto-injectors, prefilled syringes or prefilled cartridges.

For some applications, it may be desired to provide one or more coatings or layers to the interior wall of a parenteral container to modify the properties of that container. For example, one or more coatings or layers may be added to a parenteral container, e.g., to improve the barrier properties of the container and prevent interaction between the container wall (or an underlying coating) and drug product held within the container. Such coatings or layers may be constructed in accordance with the teachings of PCT Application PCT/US2014/023813, filed on Mar. 11, 2014, which is incorporated by reference herein in its entirety.

For example, as shown in FIG. 5A, which is a first alternative embodiment of an enlarged sectional view of the medical barrel 12 of the syringe assembly 10 of FIG. 5, the inner surface 14 of the medical barrel 12 may include a coating set 400 comprising one or more coatings or layers. The medical barrel 12 may include at least one tie coating or layer 402, at least one barrier coating or layer 404, and at least one organo-siloxane coating or layer 406. The organo-siloxane coating or layer 406 preferably has pH protective properties. This embodiment of the coating set 400 is referred to herein as a “tri-layer coating set” in which the barrier coating or layer 404 is protected against contents having a pH otherwise high enough to remove it by being sandwiched between the pH protective organo-siloxane coating or layer 406 and the tie coating or layer 402. The contemplated thicknesses of the respective layers in nanometers (preferred ranges in parentheses) are given in the following Tri-layer Thickness Table:

Tri-layer Thickness Table

| Adhesion (nm) | Barrier (nm) | Protection (nm) |
|---|---|---|
| 5-100 | 20-200 | 50-500 |
| (5-20) | (20-30) | (100-200) |

Properties and compositions of each of the coatings that make up the tri-layer coating set are now described.

The tie coating or layer 402 has at least two functions. One function of the tie coating or layer 402 is to improve adhesion of a barrier coating or layer 404 to a substrate (e.g., the inner surface 14 of the barrel 12), in particular a thermoplastic substrate, although a tie layer can be used to improve adhesion to a glass substrate or to another coating or layer. For example, a tie coating or layer, also referred to as an adhesion layer or coating can be applied to the substrate and the barrier layer can be applied to the adhesion layer to improve adhesion of the barrier layer or coating to the substrate.

Another function of the tie coating or layer 402 has been discovered: a tie coating or layer 402 applied under a barrier coating or layer 404 can improve the function of a pH protective organo-siloxane coating or layer 406 applied over the barrier coating or layer 404.

The tie coating or layer 402 can be composed of, comprise, or consist essentially of SiOₓC_{y}, in which x is between 0.5 and 2.4 and y is between 0.6 and 3. Alternatively, the atomic ratio can be expressed as the formula SiwOxCy. The atomic ratios of Si, O, and C in the tie coating or layer 402 are, as several options:

Si 100: O 50-150: C 90-200 (i.e. w=1, x=0.5 to 1.5, y=0.9 to 2);

Si 100: O 70-130: C 90-200 (i.e. w=1, x=0.7 to 1.3, y=0.9 to 2)

Si 100: O 80-120: C 90-150 (i.e. w=1, x=0.8 to 1.2, y=0.9 to 1.5)

Si 100: O 90-120: C 90-140 (i.e. w=1, x=0.9 to 1.2, y=0.9 to 1.4), or

Si 100: O 92-107: C 116-133 (i.e. w=1, x=0.92 to 1.07, y=1.16 to 1.33).

The atomic ratio can be determined by XPS. Taking into account the H atoms, which are not measured by XPS, the tie coating or layer 402 may thus in one aspect have the formula SiwOxCyHz (or its equivalent SiOxCy), for example where w is 1, x is from about 0.5 to about 2.4, y is from about 0.6 to about 3, and z is from about 2 to about 9. Typically, a tie coating or layer 402 would hence contain 36% to 41% carbon normalized to 100% carbon plus oxygen plus silicon.

The barrier coating or layer 404 for any embodiment defined in this specification (unless otherwise specified in a particular instance) is a coating or layer, optionally applied by PECVD as indicated in U.S. Pat. No. 7,985,188. The barrier coating preferably is characterized as a “SiOx” coating, in which x, the ratio of oxygen to silicon atoms, is from about 1.5 to about 2.9. The thickness of the SiOx or other barrier coating or layer can be measured, for example, by transmission electron microscopy (TEM), and its composition can be measured by X-ray photoelectron spectroscopy (XPS). The barrier layer is effective to prevent oxygen, carbon dioxide, water vapor, or other gases (e.g. residual monomers of the polymer from which the container wall is made) from entering the container and/or to prevent leaching of the pharmaceutical material into or through the container wall.

Preferred methods of applying the barrier layer 404 and tie layer 402 to the inner surface 14 of the barrel 12 is by plasma enhanced chemical vapor deposition (PECVD), such as described in, e.g., U.S. Pat. App. Pub. No. 20130291632, which is incorporated by reference herein in its entirety.

The Applicant has found that barrier layers or coatings of SiOx are eroded or dissolved by some fluids, for example aqueous compositions having a pH above about 5. Since coatings applied by chemical vapor deposition can be very thin—tens to hundreds of nanometers thick—even a relatively slow rate of erosion can remove or reduce the effectiveness of the barrier layer in less time than the desired shelf life of a product package. This is particularly a problem for fluid pharmaceutical compositions, since many of them have a pH of roughly 7, or more broadly in the range of 5 to 9, similar to the pH of blood and other human or animal fluids. The higher the pH of the pharmaceutical preparation, the more quickly it erodes or dissolves the SiOx coating. Optionally, this problem can be addressed by protecting the barrier coating or layer, or other pH sensitive material, with a pH protective organo-siloxane coating or layer.

Optionally, the pH protective coating or layer 406 can be composed of, comprise, or consist essentially of SiwOxCyHz (or its equivalent SiOxCy) or SiwNxCyHz or its equivalent SiNxCy). The atomic ratio of Si:O:C or Si:N:C can be determined by XPS (X-ray photoelectron spectroscopy). Taking into account the H atoms, the pH protective coating or layer may thus in one aspect have the formula SiwOxCyHz, or its equivalent SiOxCy, for example where w is 1, x is from about 0.5 to about 2.4, y is from about 0.6 to about 3, and z is from about 2 to about 9.

Typically, expressed as the formula SiwOxCy, the atomic ratios of Si, O, and C are, as several options:

Si 100: O 50-150: C 90-200 (i.e. w=1, x=0.5 to 1.5, y=0.9 to 2);

Si 100: O 70-130: C 90-200 (i.e. w=1, x=0.7 to 1.3, y=0.9 to 2)

Si 100: O 80-120: C 90-150 (i.e. w=1, x=0.8 to 1.2, y=0.9 to 1.5)

Si 100: O 90-120: C 90-140 (i.e. w=1, x=0.9 to 1.2, y=0.9 to 1.4)

Si 100: O 92-107: C 116-133 (i.e. w=1, x=0.92 to 1.07, y=1.16 to 1.33),

or

Si 100: O 80-130: C 90-150.

Alternatively, the organo-siloxane coating or layer can have atomic concentrations normalized to 100% carbon, oxygen, and silicon, as determined by X-ray photoelectron spectroscopy (XPS) of less than 50% carbon and more than 25% silicon. Alternatively, the atomic concentrations are from 25 to 45% carbon, 25 to 65% silicon, and 10 to 35% oxygen. Alternatively, the atomic concentrations are from 30 to 40% carbon, 32 to 52% silicon, and 20 to 27% oxygen. Alternatively, the atomic concentrations are from 33 to 37% carbon, 37 to 47% silicon, and 22 to 26% oxygen.

Optionally, the atomic concentration of carbon in the pH protective coating or layer 406, normalized to 100% of carbon, oxygen, and silicon, as determined by X-ray photoelectron spectroscopy (XPS), can be greater than the atomic concentration of carbon in the atomic formula for the organosilicon precursor. For example, embodiments are contemplated in which the atomic concentration of carbon increases by from 1 to 80 atomic percent, alternatively from 10 to 70 atomic percent, alternatively from 20 to 60 atomic percent, alternatively from 30 to 50 atomic percent, alternatively from 35 to 45 atomic percent, alternatively from 37 to 41 atomic percent.

Optionally, the atomic ratio of carbon to oxygen in the pH protective coating or layer 406 can be increased in comparison to the organosilicon precursor, and/or the atomic ratio of oxygen to silicon can be decreased in comparison to the organosilicon precursor.

An exemplary empirical composition for a pH protective coating according to an optional embodiment is SiO_{1.3}C_{0.8}H_{3.6}.

Optionally in any embodiment, the pH protective coating or layer 406 comprises, consists essentially of, or consists of PECVD applied coating.

Optionally in any embodiment, the pH protective coating or layer 406 is applied by employing a precursor comprising, consisting essentially of, or consisting of a silane. Optionally in any embodiment, the silane precursor comprises, consists essentially of, or consists of any one or more of an acyclic or cyclic silane, optionally comprising, consisting essentially of, or consisting of any one or more of silane, trimethylsilane, tetramethylsilane, Si2-Si4 silanes, triethyl silane, tetraethyl silane, tetrapropylsilane, tetrabutylsilane, or octamethylcyclotetrasilane, or tetramethylcyclotetrasilane.

Optionally in any embodiment, the pH protective coating or layer 406 comprises, consists essentially of, or consists of PECVD applied amorphous or diamond-like carbon. Optionally in any embodiment, the amorphous or diamond-like carbon is applied using a hydrocarbon precursor. Optionally in any embodiment, the hydrocarbon precursor comprises, consists essentially of, or consists of a linear, branched, or cyclic alkane, alkene, alkadiene, or alkyne that is saturated or unsaturated, for example acetylene, methane, ethane, ethylene, propane, propylene, n-butane, i-butane, butane, propyne, butyne, cyclopropane, cyclobutane, cyclohexane, cyclohexene, cyclopentadiene, or a combination of two or more of these. Optionally in any embodiment, the amorphous or diamond-like carbon coating has a hydrogen atomic percent of from 0.1% to 40%, alternatively from 0.5% to 10%, alternatively from 1% to 2%, alternatively from 1.1 to 1.8%

Optionally in any embodiment, the pH protective coating or layer 406 comprises, consists essentially of, or consists of PECVD applied SiN. Optionally in any embodiment, the PECVD applied SiN is applied using a silane and a nitrogen-containing compound as precursors. Optionally in any embodiment, the silane is an acyclic or cyclic silane, optionally comprising, consisting essentially of, or consisting of silane, trimethylsilane, tetramethylsilane, Si2-Si4 silanes, triethylsilane, tetraethylsilane, tetrapropylsilane, tetrabutylsilane, octamethylcyclotetrasilane, or a combination of two or more of these. Optionally in any embodiment, the nitrogen-containing compound comprises, consists essentially of, or consists of any one or more of: nitrogen gas, nitrous oxide, ammonia or a silazane. Optionally in any embodiment, the silazane comprises, consists essentially of, or consists of a linear silazane, for example hexamethylene disilazane (HMDZ), a monocyclic silazane, a polycyclic silazane, a polysilsesquiazane, or a combination of two or more of these.

Optionally in any embodiment, the PECVD for the pH protective coating or layer 406 is carried out in the substantial absence or complete absence of an oxidizing gas. Optionally in any embodiment, the PECVD for the pH protective coating or layer 406 is carried out in the substantial absence or complete absence of a carrier gas.

Optionally an FTIR absorbance spectrum of the pH protective coating or layer 406 SiOxCyHz has a ratio greater than 0.75 between the maximum amplitude of the Si—O—Si symmetrical stretch peak normally located between about 1000 and 1040 cm-1, and the maximum amplitude of the Si—O—Si asymmetric stretch peak normally located between about 1060 and about 1100 cm-1. Alternatively in any embodiment, this ratio can be at least 0.8, or at least 0.9, or at least 1.0, or at least 1.1, or at least 1.2. Alternatively in any embodiment, this ratio can be at most 1.7, or at most 1.6, or at most 1.5, or at most 1.4, or at most 1.3. Any minimum ratio stated here can be combined with any maximum ratio stated here, as an alternative embodiment.

Optionally, in any embodiment the pH protective coating or layer 406, in the absence of the liquid filling, has a non-oily appearance. This appearance has been observed in some instances to distinguish an effective pH protective coating or layer 406 from a lubricity layer (e.g., as described in U.S. Pat. No. 7,985,188), which in some instances has been observed to have an oily (i.e. shiny) appearance.

The pH protective coating or layer optionally can be applied by plasma enhanced chemical vapor deposition (PECVD) of a precursor feed comprising an acyclic siloxane, a monocyclic siloxane, a polycyclic siloxane, a polysilsesquioxane, a monocyclic silazane, a polycyclic silazane, a polysilsesquiazane, a silatrane, a silquasilatrane, a silproatrane, an azasilatrane, an azasilquasiatrane, an azasilproatrane, or a combination of any two or more of these precursors. Some particular, non-limiting precursors contemplated for such use include octamethylcyclotetrasiloxane (OMCTS).

Other precursors and methods can be used to apply the pH protective coating or layer 406 or passivating treatment. For example, hexamethylene disilazane (HMDZ) can be used as the precursor. HMDZ has the advantage of containing no oxygen in its molecular structure. This passivation treatment is contemplated to be a surface treatment of the SiOx barrier layer with HMDZ. To slow down and/or eliminate the decomposition of the silicon dioxide coatings at silanol bonding sites, the coating must be passivated. It is contemplated that passivation of the surface with HMDZ (and optionally application of a few mono layers of the HMDZ-derived coating) will result in a toughening of the surface against dissolution, resulting in reduced decomposition. It is contemplated that HMDZ will react with the —OH sites that are present in the silicon dioxide coating, resulting in the evolution of NH₃ and bonding of S—(CH₃)₃ to the silicon (it is contemplated that hydrogen atoms will be evolved and bond with nitrogen from the HMDZ to produce NH₃).

Another way of applying the pH protective coating or layer is to apply as the pH protective coating or layer an amorphous carbon or fluorocarbon coating, or a combination of the two.

Amorphous carbon coatings can be formed by PECVD using a saturated hydrocarbon, (e.g. methane or propane) or an unsaturated hydrocarbon (e.g. ethylene, acetylene) as a precursor for plasma polymerization. Fluorocarbon coatings can be derived from fluorocarbons (for example, hexafluoroethylene or tetrafluoroethylene). Either type of coating, or a combination of both, can be deposited by vacuum PECVD or atmospheric pressure PECVD. It is contemplated that that an amorphous carbon and/or fluorocarbon coating will provide better passivation of an SiOx barrier layer than a siloxane coating since an amorphous carbon and/or fluorocarbon coating will not contain silanol bonds.

It is further contemplated that fluorosilicon precursors can be used to provide a pH protective coating or layer over a SiOx barrier layer. This can be carried out by using as a precursor a fluorinated silane precursor such as hexafluorosilane and a PECVD process. The resulting coating would also be expected to be a non-wetting coating.

Yet another coating modality contemplated for protecting or passivating a SiOx barrier layer is coating the barrier layer using a polyamidoamine epichlorohydrin resin. For example, the barrier coated part can be dip coated in a fluid polyamidoamine epichlorohydrin resin melt, solution or dispersion and cured by autoclaving or other heating at a temperature between 60 and 100° C. It is contemplated that a coating of polyamidoamine epichlorohydrin resin can be preferentially used in aqueous environments between pH 5-8, as such resins are known to provide high wet strength in paper in that pH range. Wet strength is the ability to maintain mechanical strength of paper subjected to complete water soaking for extended periods of time, so it is contemplated that a coating of polyamidoamine epichlorohydrin resin on a SiOx barrier layer will have similar resistance to dissolution in aqueous media. It is also contemplated that, because polyamidoamine epichlorohydrin resin imparts a lubricity improvement to paper, it will also provide lubricity in the form of a coating on a thermoplastic surface made of, for example, COC or COP.

Even another approach for protecting a SiOx layer is to apply as a pH protective coating or layer a liquid-applied coating of a polyfluoroalkyl ether, followed by atmospheric plasma curing the pH protective coating or layer. For example, it is contemplated that the process practiced under the trademark TriboGlide® can be used to provide a pH protective coating or layer 406 that also provides lubricity.

Thus, a pH protective coating for a thermoplastic syringe wall according to an aspect of the invention may comprise, consist essentially of, or consist of any one of the following: plasma enhanced chemical vapor deposition (PECVD) applied coating having the formula SiOxCyHz, in which x is from 0 to 0.5, alternatively from 0 to 0.49, alternatively from 0 to 0.25 as measured by X ray photoelectron spectroscopy (XPS), y is from about 0.5 to about 1.5, alternatively from about 0.8 to about 1.2, alternatively about 1, as measured by XPS, and z is from 0 to 2 as measured by Rutherford Backscattering Spectrometry (RBS), alternatively by Hydrogen Forward Scattering Spectrometry (HFS); or PECVD applied amorphous or diamond-like carbon, CHz, in which z is from 0 to 0.7, alternatively from 0.005 to 0.1, alternatively from 0.01 to 0.02; or PECVD applied SiNb, in which b is from about 0.5 to about 2.1, alternatively from about 0.9 to about 1.6, alternatively from about 1.2 to about 1.4, as measured by XPS.

PECVD apparatus suitable for applying any of the PECVD coatings or layers described in this specification, including the tie coating or layer, the barrier coating or layer or the organo-siloxane coating or layer, is shown and described in U.S. Pat. No. 7,985,188 and U.S. Pat. App. Pub. No. 20130291632. This apparatus optionally includes a vessel holder, an inner electrode, an outer electrode, and a power supply. A vessel seated on the vessel holder defines a plasma reaction chamber, optionally serving as its own vacuum chamber. Optionally, a source of vacuum, a reactant gas source, a gas feed or a combination of two or more of these can be supplied. Optionally, a gas drain, not necessarily including a source of vacuum, is provided to transfer gas to or from the interior of a vessel seated on the port to define a closed chamber.

It is contemplated that syringes having a plunger-contacting inner surface are provided substantially without the presence of a flowable lubricant. As used herein, “substantially without the presence of a flowable lubricant,” means that a flowable lubricant (e.g., PDMS) is not provided to a syringe barrel in amounts that would contribute to the lubricity of the plunger-syringe system. Since it is sometimes the practice to use a flowable lubricant when handling plungers prior to assembling them into syringes, “substantially without the presence of a flowable lubricant” in some cases may contemplate the presence of trace amounts of such lubricant as a result of such handling practices.

Accordingly, in one optional aspect, the invention may incorporate an organo-siloxane coating on the inner surface of a parenteral container which provides lubricious properties conducive to acceptable plunger operation. The organo-siloxane coating may, for example, be any embodiment of the pH protective coating discussed above. The organo-siloxane coating may be applied directly to the interior wall of the container or as a top layer on a multi-layer coating set, e.g., the tri-layer coating set discussed above.

The organo-siloxane coating can optionally provide multiple functions: (1) a pH resistant layer that protects an underlying layer or underlying polymer substrate from drug products having a pH from 4-10, optionally from 5-9; (2) a drug contact surface that minimizes aggregation, extractables and leaching; (3) in the case of a protein-based drug, reduced protein binding on the container surface; and (4) a lubricating layer, e.g., to facilitate plunger advancement when dispensing contents of a syringe.

Use of an organo-siloxane coating on a polymer-based container as the contact surface for a plunger provides distinct advantages. Plastic syringes and cartridges may be injection molded to tighter tolerances than their glass counterparts. It is contemplated that the dimensional precision achievable through injection molding allows optimization of the inside diameter of a syringe to provide sufficient compression to the plunger for CCI and gas-tightness on the one hand, while not over-compressing the plunger so as to provide desired plunger force upon administration of the drug product. Optimally, this would eliminate or dramatically reduce the need for lubricating the syringe or cartridge with a flowable lubricant.

Lubricity coatings, e.g., prepared according to methods disclosed in U.S. Pat. No. 7,985,188 (incorporated by reference herein in its entirety), are particularly well suited to provide a desired level of lubricity for plungers in parenteral containers. Such lubricity coatings are preferably applied using plasma enhanced chemical vapor deposition (“PECVD”) and may have one of the following atomic ratios, SiwOxCy or SiwNxCy, where w is 1, x is from about 0.5 to 2.4 and y is from about 0.6 to about 3. Such lubricity coatings may have a thickness between 10 and 500 nm. Advantages of such plasma coated lubricity layers may include lower migratory potential to move into the drug product or patient than liquid, sprayed or micron-coated silicones. It is contemplated that use of such lubricity coatings to reduce plunger force is within the broad scope of the invention. Optionally, as shown in FIG. 5B, a PECVD lubricity coating 408 may be disposed on top of a tri-layer coating set, making a four layer coating set.

The PECVD coating apparatus and process are as described generally in PECVD protocols of U.S. Pat. No. 7,985,188, or PCT/US16/47622. The entire text and drawings of U.S. Pat. No. 7,985,188 and PCT/US16/47622 are incorporated here by reference.

In one embodiment, the tie or adhesion coating or layer and the barrier coating or layer, and optionally the pH protective layer, are applied in the same apparatus, without breaking vacuum between the application of the adhesion coating or layer and the barrier coating or layer or, optionally, between the barrier coating or layer and the pH protective coating or layer. During the process, a partial vacuum is drawn in the lumen. While maintaining the partial vacuum unbroken in the lumen, a tie coating or layer of SiOxCy is applied by a tie PECVD coating process. The tie PECVD coating process is carried out by applying sufficient power to generate plasma within the lumen while feeding a gas suitable for forming the coating. The gas feed includes a linear siloxane precursor, optionally oxygen, and optionally an inert gas diluent. The values of x and y are as determined by X-ray photoelectron spectroscopy (XPS). Then, while maintaining the partial vacuum unbroken in the lumen, the plasma is extinguished. A tie coating or layer of SiOxCy, for which x is from about 0.5 to about 2.4 and y is from about 0.6 to about 3, is produced on the inside surface as a result.

Later during the process, while maintaining the partial vacuum unbroken in the lumen, a barrier coating or layer is applied by a barrier PECVD coating process. The barrier PECVD coating process is carried out by applying sufficient power to generate plasma within the lumen while feeding a gas. The gas feed includes a linear siloxane precursor and oxygen. A barrier coating or layer of SiOx, wherein x is from 1.5 to 2.9 as determined by XPS is produced between the tie coating or layer and the lumen as a result.

Then optionally, while maintaining the partial vacuum unbroken in the lumen, the plasma is extinguished.

Later, as a further option, a pH protective coating or layer of SiOxCy can be applied. In this formula as well, x is from about 0.5 to about 2.4 and y is from about 0.6 to about 3, each as determined by XPS. The pH protective coating or layer is optionally applied between the barrier coating or layer and the lumen, by a pH protective PECVD coating process. This process includes applying sufficient power to generate plasma within the lumen while feeding a gas including a linear siloxane precursor, optionally oxygen, and optionally an inert gas diluent.

Then optionally, while maintaining the partial vacuum unbroken in the lumen, the plasma is extinguished.

Later, as a further option, a lubricity coating or layer of SiOxCy can be applied. In this formula as well, x is from about 0.5 to about 2.4 and y is from about 0.6 to about 3, each as determined by XPS. The lubricity coating or layer is optionally applied on top of the pH protective coating, by a lubricity PECVD coating process. This process includes applying sufficient power to generate plasma within the lumen while feeding a gas including an organo siloxane precursor, optionally oxygen, and optionally an inert gas diluent.

Optionally in any embodiment, the PECVD process for applying the tie coating or layer, the barrier coating or layer, and/or the pH protective coating or layer, and/or the lubricty coating or any combination of two or more of these, is carried out by applying pulsed power (alternatively the same concept is referred to in this specification as “energy”) to generate plasma within the lumen.

Alternatively, the tie PECVD coating process, or the barrier PECVD coating process, or the pH protective PECVD coating process, or any combination of two or more of these, can be carried out by applying continuous power to generate plasma within the lumen.

### Trilayer Coating Process Protocol (all Layers Coated in the Same Apparatus)

The trilayer coating as described in this embodiment is applied by adjusting the flows of a single organosilicon monomer (HMDSO) and oxygen and also varying the PECVD generating power between each layer (without breaking vacuum between any two layers).

The vessel (e.g., a COC syringe) is placed on a vessel holder, sealed, and a vacuum is pulled within the vessel. After pulling vacuum, the gas feed of precursor, oxygen, and argon is introduced, then at the end of the “plasma delay” continuous (i.e. not pulsed) RF power at 13.56 MHz is turned on to form the tie coating or layer. Then power is turned off, gas flows are adjusted, and after the plasma delay power is turned on for the second layer—an SiOx barrier coating or layer. This is then repeated for a third layer before the gases are cut off, the vacuum seal is broken, and the vessel is removed from the vessel holder. The layers are put down in the order of Tie then Barrier then pH Protective. An exemplary process settings are as shown in the following table:

| | | | | | Deposition |
|---|---|---|---|---|---|
| | O₂ | Ar | HMDSO | Power | Time |
| Coating | (sccm) | (sccm) | (sccm) | (W) | (sec) |
| Tie | 1 | 40 | 2 | 20 | 2.5 |
| Barrier | 100 | 0 | 1 | 60 | 15 |
| pH Protective | 1 | 40 | 2 | 20 | 10 |

As a still a still further alternative, pulsed power can be used for some steps, and continuous power can be used for others. For example, when preparing a trilayer coating or layer composed of a tie coating or layer, a barrier coating or layer, and a pH protective coating or layer, an option specifically contemplated for the tie PECVD coating process and for the pH protective PECVD coating process is pulsed power, and an option contemplated for the corresponding barrier layer is using continuous power to generate plasma within the lumen.

### Optional Injectable Product Compositions

Optionally, in any embodiment, syringes according to the disclosed concept are prefilled with an injectable drug product.

Optionally in any embodiment, the injectable drug product may be an ophthalmic drug suitable for intravitreal injection. Optionally in any embodiment, the ophthalmic drug comprises a VEGF antagonist, optionally an anti-VEGF antibody or an antigen-binding fragment of such antibody. Optionally in any embodiment, the VEGF antagonist comprises Ranibizumab, Aflibercept, or a combination of these.

Optionally in any embodiment, the concentration of the liquid formulation of an ophthalmic drug suitable for intravitreal injection is 1 to 100 mg of the drug active agent per ml. of the liquid formulation 40 (mg/ml), alternatively 2-75 mg/ml, alternatively 3-50 mg/ml, alternatively 5 to 30 mg/ml, and alternatively 6 or 10 mg/ml.

Optionally in any embodiment, the liquid formulation of an ophthalmic drug suitable for intravitreal injection comprises 6 mg/mL, alternatively 10 mg/mL, of Ranibizumab.

Optionally in any embodiment, the ophthalmic drug suitable for intravitreal injection further comprises: a buffer in an amount effective to provide a pH of the liquid formulation 40 in the range from about 5 to about 7; a non-ionic surfactant in the range of 0.005 to 0.02% mg./mL of complete formulation, alternatively in the range of 0.007 to 0.018% mg./mL of complete formulation, alternatively in the range of 0.008 to 0.015% mg./mL of complete formulation, alternatively in the range of 0.009 to 0.012% mg./mL of complete formulation, alternatively in the range of 0.009 to 0.011% mg./mL of complete formulation, alternatively 0.01% mg./mL of complete formulation; and water for injection.

Optionally in any embodiment, the ophthalmic drug suitable for intravitreal injection comprises 6 mg/mL, alternatively 0 mg/mL, of Ranibizumab; 100 mg/mL of α, α-trehalose dihydrate, 1.98 mg/mL L-histidine; and 0.1 mg/mL Polysorbate 20 in water for injection.

### Industry Standards for Testing Aspects of Plunger

Testing of compression setting properties of the plunger assembly may be conducted using methods known in the art, for example, ASTM D395.

Testing of adhesive properties or bonding strength between a film (e.g., fluoropolymer) and the plunger may be conducted using methods known in the art, for example, according to ASTM D1995-92 (2011) or D1876-08.

Plunger sliding force is the force required to maintain movement of a plunger in a syringe or cartridge barrel, for example during aspiration or dispense. It can advantageously be determined using, e.g., the ISO 7886-1:1993 test known in the art, or to the currently pending published test method to be incorporated into ISO 11040-4. Plunger breakout force, which may be tested using the same method as that for testing plunger sliding force, is the force required to start a stationary plunger moving within a syringe or cartridge barrel. Machinery useful in testing plunger sliding and breakout force is, e.g., an Instron machine using a 50 N transducer.

Testing for extractables, i.e., amount of material that migrates from the plunger into the liquid within the syringe or cartridge, may be conducted using methods set forth in Ph. Eur. 2.9.17 Test for Extractable Volume of Parenteral Preparations, for example.

Testing of container closure integrity (CCI) may be done using a vacuum decay leak detection method, wherein a vacuum is maintained inside of a test volume and pressure rise is measured over time. A large enough pressure rise is an indication that there is flow into the system, which is evidence of a leak. Optionally, the vacuum decay test is implemented over two separate cycles. The first cycle is dedicated to detecting large leaks over a very short duration. A relatively weak vacuum is pulled for the first cycle because if a gross leak is detected, a large pressure differential is not necessary to detect a large pressure rise. Use of a first cycle as described helps to shorten total test time if a gross leak exists. If no leak is detected in the first cycle, a second cycle is run, which complies with ASTM F2338-09 Standard Test Method for Nondestructive Detection of Leaks in Packages by Vacuum Decay Method. The second cycle starts out with a system evaluation to lower the signal to noise ratio in the pressure rise measurements. A relatively strong vacuum is pulled for a long period of time in the second cycle to increase the chance of detecting a pressure rise in the system.

Testing of air leakage past the syringe piston during aspiration may be conducted using methods known in the art, for example, ISO 7886-1:1993.

Testing of liquid leakage at a syringe piston under compression may be conducted using methods known in the art, for example, ISO 7886-1:2015, Annex B for liquid leakage, with blocked fluid path, by applying an axial force on the plunger stopper by final plunger rod, consistent with the maximum force generated during use.

In an exemplary method of applying this standard, a 0.5 mL syringe may be filled with 0.165 mL MILLI-Q high purity water. Plungers, optionally West FLUROTEC plungers are vacuum loaded into the filled syringes. Plunger assemblies with axial protrusions are disposed within the plungers as described in this specification to place plungers into storage mode. The crosshead compresses at a rate of 10 mm/min until reaching a maximum force of 5.43 N, which corresponds to 300 kPa pressure in the syringe (or a force consistent with the maximum force generated during use). The crosshead makes small adjustments to hold at the maximum force for 30 seconds. In this implementation, the ISO 7886-1 test is considered failed if any water from inside the syringe moves back past any rib on the plunger.

Various aspects of the invention will be illustrated in more detail with reference to the following Examples, but it should be understood that the present invention is not deemed to be limited thereto.

### EXAMPLES

### Example 1

### Impact of Axial Length of the Protrusion on Fᵢ after Aging

In this example, three groups of syringes A, B and C made of COP were the subject of an experiment to determine the effect of axial protrusion length on break loose force (Fi) after aging. Each group had 5 syringes. Group A had no axial protrusion on the plunger rod (which thus pushes the plunger at the plunger's proximal end instead of within the inner cavity of the plunger sleeve). Group B had the configuration shown in FIGS. 5 and 6, with an axial protrusion having an axial length of 5.71 mm. Group C had the configuration shown in FIGS. 5 and 6, with an axial protrusion having an axial length of 9.1 mm. The only difference for the three configurations is the axial length (L) of the protrusion. For all three groups of syringes, the plungers were identical. The axial length of the inner cavity of each plunger was 5.3 mm. This measurement represents the length from the proximal end of the plunger sleeve to the distal-most section of the interior surface (engagement surface), while the plunger is in its natural state (i.e., not radially or axially compressed or stretched).

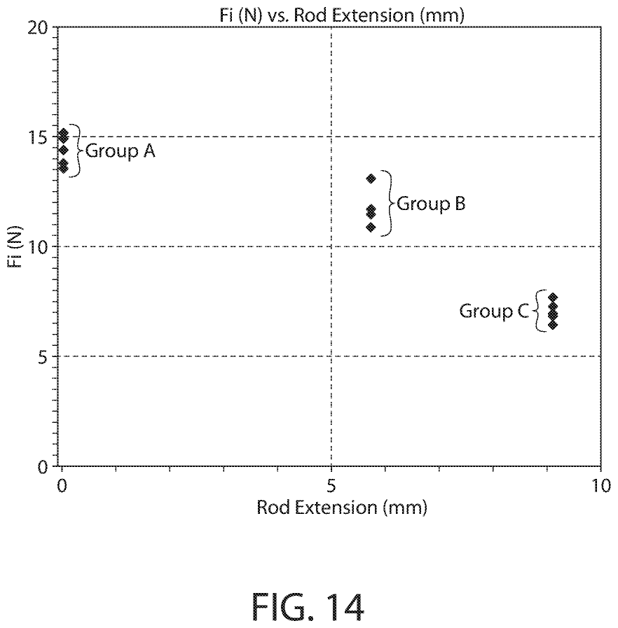

All of the barrels of the syringes were coated with a trilayer coating set with an OMCTS lubricity coating disposed on the trilayer coating set, thereby providing a four layer coating set, as disclosed in this specification (see FIG. 5B). These syringes were filled with a buffer solution (aqueous solution with 10 mM histidine HCl, 10% α, α-trehalose dehydrate, 0.01% polysorbate, pH 5.5). The filled syringes were stored at 4° C. After 9 months of storage, these syringes were tested at 190 mm/min for their break loose forces without needles. The data of break loose force depending on axial protrusion length are shown in FIG. 14.

When these syringes were tested for break loose force, the plunger rod moved axially from the proximal end to the distal end within the barrel. For a syringe of Group A, during the testing, the absence of a protrusion resulted in an axial compression force exerted completely onto the proximal end of the plunger by moving the plunger rod in a distal direction. For a syringe of Group B, an axial protrusion which was a little longer than the axial length of the inner cavity resulted in an axial compression force exerted onto the proximal end of the plunger while the plunger was stretched by the protrusion at the distal end of the plunger from the inside. For a syringe of Group C, an axial protrusion much longer than the axial length of the inner cavity resulted in a noticeable gap between the proximal end of the plunger and the distal end of the plunger rod, when the plunger was moved from a rest position. Therefore, for Group C, the plunger was stretched at the distal end of the inner cavity by the protrusion from the inside with no compression force exerted on the plunger at the proximal end thereof. The data show that the syringes of Group C afforded the lowest break loose forces, the syringes of Group A afforded the highest break loose forces and the syringes of Group B were in-between.

### Example 2

### Impact of Axial Length of the Protrusion on Fᵢ after Aging

In this example, syringes were PECVD coated with a four layer coating set on the barrels' inner walls by the process as described in the specification and illustrated in FIG. 5B. The syringes were filled with 1.165 mL of high-purity water and vacuum loaded with plungers. The plungers were part of plunger assemblies separated into the following groups: A1, B1, C1 and D1. Each plunger (West 4023/50) is made from a bromobutyl rubber with a durometer of 50, which is covered with a fluoropolymer (e.g. ETFE) film on the drug contact surface, such as the nose cone region. These syringes and plunger assemblies were tested for their break loose forces (Fᵢ) after 1 day, 7 days and 28 days of storage, respectively. The configurations of the plunger assemblies corresponding to the four syringes were as follows:

A1: no protrusion (length=0.0 mm);

B1: protrusion (length=4.7 mm);

C1: protrusion (length=5.2 mm);

D1: protrusion with (length=5.7 mm).

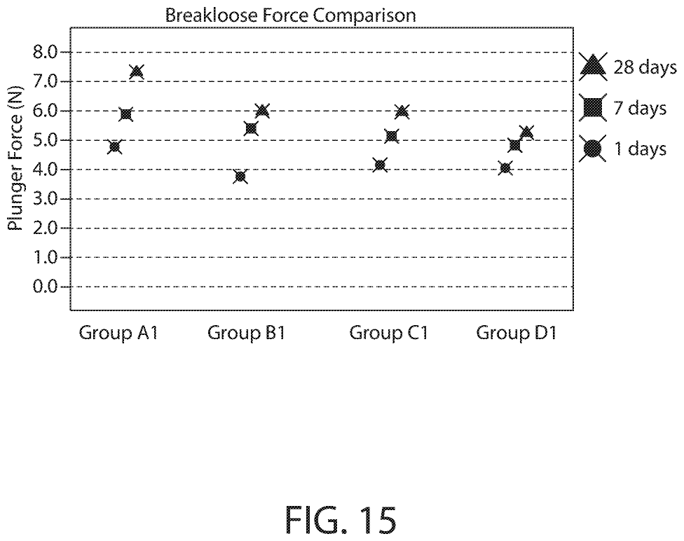

For groups B1, C1 and D1, aside from respective lengths, the axial protrusion was similar to the design of FIG. 5. The testing results are shown in Table 1, below and FIG. 15. The data demonstrate that the syringe with a protrusion longer than the inner cavity of the plunger (i.e. D1) affords the lowest break out force Fᵢ. The syringe with no protrusion (A1) gives the highest break out force Fᵢ, since the plunger does not stretch at all and is compressed from behind. The syringes with protrusions shorter than the inner cavity of the plungers (i.e. B1 and C1) provide results landing in-between. The following table sets forth the results of this test wherein the numbers represent mean break loose force (N).

| Aging | | | | |
|---|---|---|---|---|
| Time | A1 | B1 | C1 | D1 |
| (days) | (L = 0 mm) | (L = 4.7 mm) | (L = 5.2 mm) | (L = 5.7 mm) |
| 1 | 4.74 | 3.69 | 4.03 | 3.90 |
| 7 | 5.83 | 5.33 | 5.01 | 4.67 |
| 28 | 7.29 | 5.94 | 5.86 | 5.07 |

These data show that the plunger assemblies comprising axial protrusions reduced break loose force on average about 20%-25% compared to the plunger rod without axial protrusions at a given time point.

### Example 3

### Burst Test with Plunger Rod Axial Protrusion

Burst testing is used to determine the maximum force required for the plunger rod extension to break through the rubber plunger (hereinafter referred to as “burst”).

The plunger assembly 20 of FIG. 5 is subjected to burst testing. The axial protrusion is made of polypropylene, is 9.1 mm long and 1.4 mm in diameter. A group of syringes are filled with 0.165 mL of water for injection (WFI) and are stoppered (i.e., plungers are inserted in to the barrels). The filled syringe is stored at 4° C. for 15 days. The syringes are removed from the refrigerator and allowed to warm to room temperature for one hour prior to testing.

The syringes are loaded onto an Instron instrument and the plunger rod is assembled with the plunger sleeve, as described in this disclosure.

The instrument pushes plunger rod at a constant rate of 190 mm/min. The force required to push the plunger is measured. When the plunger rod makes contact with the underside of the plunger, the force begins to increase. The data show that the plunger rod extension stretches the rubber plunger approximately 1.5 mm before burst. The amount of force required for burst is −25N. This test demonstrates that when utilizing preferred plunger and plunger rod materials at preferred dimensions, it is desirable to limit rod extension length so that the rubber plunger cannot be elongated more than 1.5 mm. This will help ensure that the plunger rod burst will not occur.

### Example 4

### Leak Testing of Plunger Assembly Having Axial Protrusion with Head

In this example, leak testing was conducted with plunger assemblies having an axial protrusion 130 comprising a head 130a as described above and shown in FIGS. 3A, 3B, 7, 8 and 12A-12C. The leak testing was performed in accordance with ISO 7886-1:2015, Annex B for liquid leakage, with blocked fluid path, as described in this specification. Heads (e.g., 130a) of various shapes and dimensions were tested. A total of 20 syringes were tested using each axial protrusion head configuration per temperature and time point. Testing was performed at storage temperatures of 4° C., 25° C. and 40° C., each at time points of 1 day, 3 days, 7 days, 1 month, 3 months, 6 months and 9 months, except that runs were not done at 6 and 9 months at 40° C. In other words, a total of 380 test runs for each axial protrusion head configuration and dimension were conducted.

It was found that the axial protrusion 130 having the head 130a precisely as depicted in FIGS. 3A, 3B, 7, 8 and 12A-12C had superior performance compared to other tested configurations. Further, it was found that the head 130a having a larger diameter (in this case 2.25 mm, compared to 2.10 mm and 2.00 mm) provided superior results. This testing demonstrated that in this configuration, the thicker head 130a (2.25 mm diameter) provided greater radial compression against the barrel wall, which made a difference. That embodiment resulted in only a single failure out of 380 test runs and that failure was at 25° C., i.e., a total failure rate of 0.26%. At the more typical refrigerated storage temperature of 4° C., that embodiment had zero fails out of 140 test runs, i.e., a 0% failure rate at that temperature. The next best was the same shape head 130a, but at a 2.10 mm diameter. That embodiment resulted in 26 failures out of 380 runs, i.e., a total failure rate of 6.8% and 5 failures out of 140 runs at 4° C., i.e., a failure rate at that temperature of 3.6%. Another embodiment of the same shape but a 2.00 mm diameter had similar results to the same shape having the 2.10 mm diameter.

By contrast, an embodiment having a bullet shape with a 2.00 mm diameter, which did not nicely conform to the inner geometry of the inner cavity, performed much more poorly. The “bullet shaped” embodiment resulted in 67 failures out of 380 runs, i.e., a total failure rate of 17.6%. That embodiment also resulted in 23 failures out of 140 runs at 4° C., i.e., a failure rate at that temperature of 16.4%.

This experiment demonstrated that for axial protrusions having heads, the head shape and dimensions can drastically impact the efficacy of the liquid seal provided by the plunger. It appears that a head geometry that substantially conforms to corresponding geometry and dimensions of the distal compartment of the inner cavity of the plunger sleeve improves seal integrity.

### Example 5

### Fᵢ Over Time with Testing of Plunger Assembly Having Axial Protrusion with Head

In this example, break loose force over testing was conducted with plunger assemblies having an axial protrusion 130 comprising a head 130a as described above and shown in FIGS. 3A, 3B, 7, 8 and 12A-12C. Heads (e.g., 130a) of various shapes and dimensions were tested. Tests were run using different shaped/dimensioned heads at different time points and at different temperatures. In particular, five syringes were tested per time point for a total of eight time points (1 day, 3 days, 7 days, 1 month, 3 months and 9 months) and each at three different temperatures (4° C., 25° C. and 40° C.). In other words, a total of 40 syringes were tested at each temperature. The barrel was coated with silicone oil for lubrication.

The syringes were filled with 0.165 mL MILLI-Q high purity water. Plungers were loaded using a vacuum loader to a vacuum pressure of 28 in Hg (65 mbar absolute pressure). This filling process resulted in a bubble of about 0.3 mm in height. The size of the bubble did not affect Fᵢ or maximum Fₘ. Before testing, the syringes stored at respective temperatures were allowed to reach room temperature.

A control group consisted of a plunger rod without an axial protrusion, i.e, a plunger rod having a distal end that directly contacts the proximal end of the plunger sleeve from storage mode through dispensing mode. The data showed, that on average, the control group trended about 2N greater in Fᵢ than the test group at a given time point, regardless of specific head shape and configuration. For example, at 9 months, the control group averaged nearly 8 N for Fᵢ while at that same time point, the plunger assemblies of the test group averaged under 6 N for Fᵢ.

This experiment demonstrates that the shape and dimensions of the embodiment that had the lowest leak test failure rate measured comparably with other subgroups in the tested population for Fᵢ. In other words, that shape and those dimensions appear to strike a good balance between seal integrity and plunger force. This experiment further demonstrates that using an axial protrusion to stretch the plunger can reduce break loose force without significantly sacrificing seal integrity.

While the invention has been described in detail and with reference to specific examples thereof, it will be apparent to one skilled in the art that various changes and modifications can be made therein without departing from the spirit and scope thereof.

What is claimed is:

1. A plunger assembly for use in a medical barrel, comprising:
a plunger rod having a distal end and a proximal end;
an axial protrusion secured to, extending from or abutting the distal end of the plunger rod; and
a plunger comprising a plunger sleeve having an exterior surface and an interior surface surrounding an inner cavity, the exterior surface comprising a distal nose cone and an outer annular wall extending proximally from the nose cone and leading to an opening at a proximal end of the plunger sleeve, the opening receiving the axial protrusion such that the axial protrusion extends into the inner cavity and contacts an engagement surface of the interior surface, the engagement surface configured to receive a force applied in a distal direction by the axial protrusion to move the plunger assembly in the distal direction when the plunger rod is moved in the distal direction;
wherein the distal end of the plunger rod does not initially contact the proximal end of the plunger sleeve when the plunger is in a pre-elongation state and wherein application of axial force in a proximal direction onto the proximal end of the plunger rod sufficient to axially displace the proximal end of the plunger rod a predetermined distance does not axially displace the plunger in the proximal direction.

2. The plunger assembly of claim 1, wherein the plunger rod and axial protrusion are provided as a single piece, of unitary construction.

3. The plunger assembly of claim 2, wherein the axial protrusion is cylindrical and of uniform diameter substantially along its entire length, wherein a cylindrical outer surface of the axial protrusion loosely contacts the interior surface of the plunger sleeve without an interference fit so that the axial protrusion may be manually pulled out of the plunger sleeve when the plunger sleeve is disposed in a medical barrel.

4. The plunger assembly of claim 1, wherein the plunger rod and axial protrusion are provided as a multi-piece assembly comprising a proximal first portion and a distal second portion, the second portion comprising the axial protrusion, wherein the first portion and second portion may be manually pulled apart at least to the predetermined distance.

5. The plunger assembly of claim 4, wherein the first portion and second portion are assembled together in a telescoping arrangement, such that they may be pulled apart to the predetermined distance whereupon they cannot be manually pulled apart any further, wherein the first portion and second portion may be collapsed until they cannot be pushed together any further.

6. The plunger assembly of claim 5, wherein one of the first portion or the second portion includes a hub having a central hollow configured to receive a shaft of the other of the first portion or the second portion.

7. The plunger assembly of claim 5, wherein when fully collapsed, application of sufficient distally directed force onto plunger rod causes the multi-piece assembly to move as a unit in the distal direction.

8. The plunger assembly of claim 4, the axial protrusion comprising, at a distal end thereof, a head having a greater cross-sectional width or diameter than that of the portion of the axial protrusion leading to the head, wherein the head contacts the engagement surface of the interior surface of the plunger sleeve.

9. The plunger assembly of claim 8, wherein the head is disposed in a distal compartment within the inner cavity, the distal compartment having a greater cross-sectional width or diameter than a narrower section of the inner cavity proximal to the distal compartment, wherein the axial protrusion cannot be readily manually pulled out of the plunger because the head is of greater diameter or cross-sectional width than the narrower section of the inner cavity.

10. The plunger assembly of claim 9, wherein the head has a geometry and dimensions that substantially conform to corresponding geometry and dimensions of the distal compartment of the inner cavity of the plunger sleeve.

11. A pre-filled syringe comprising:
a medical barrel having an inner wall and product containing area, the product containing area having disposed therein an injectable product, the medical barrel having a distal dispensing end for dispensing the injectable product and an open proximal end configured for receipt of a plunger assembly; and
the plunger assembly according to claim 1, wherein the plunger is disposed within the medical barrel such that the nose cone faces the injectable product and at least a portion of the plunger rod extends proximally from the open proximal end of the medical barrel.

12. The pre-filled syringe of claim 11, wherein application of sufficient force onto the plunger rod in the distal direction causes the plunger assembly to displace distally down the medical barrel.

13. The pre-filled syringe of claim 12, the plunger comprising a stretch zone that is adapted to undergo elongation along a central axis of the plunger upon application of a force in the distal direction by the axial protrusion onto the engagement surface, wherein the elongation reduces an outer profile of the outer annular wall along the stretch zone.

14. The pre-filled syringe of claim 13, wherein the plunger rod and axial protrusion are configured such that the plunger rod does not contact the proximal end of the plunger sleeve when the assembly is advanced in the distal direction down the medical barrel, so as not to axially compress the plunger during actuation.

15. The pre-filled syringe of claim 11, wherein application of axial force in the proximal direction onto the proximal end of the plunger rod, sufficient to axially displace at least part of the plunger rod the predetermined distance in the proximal direction, does not cause the plunger to axially displace in the proximal direction.

16. The pre-filled syringe of claim 15, wherein the plunger rod and axial protrusion are provided as a single piece, of unitary construction and wherein application of axial force in the proximal direction onto the proximal end of the plunger rod sufficient to axially displace the plunger rod in the proximal direction to the predetermined distance, removes the axial protrusion from the plunger sleeve.

17. The pre-filled syringe of claim 11, wherein the syringe is a 0.5 mL syringe.

18. The pre-filled syringe of claim 11, wherein the medical barrel is injection molded from a clear polymer, optionally COP or COC.

19. The pre-filled syringe of claim 11, the inner wall of the medical barrel comprising a plasma enhanced chemical vapor deposition (PECVD) coating or coating set.

20. The prefilled syringe of claim 19, wherein the PECVD coating or coating set is selected from the group consisting of:
a bilayer coating set comprising a tie layer and a SiOx barrier layer disposed on the tie layer;
a trilayer coating set comprising a tie layer, an SiOx barrier layer disposed on the tie layer and an organo-siloxane layer disposed on the SiOx barrier layer; and
a four layer coating set comprising a tie layer, an SiOx barrier layer disposed on the tie layer, an organo-siloxane layer disposed on the SiOx barrier layer and a lubricity layer disposed on the organo-siloxane layer.

21. The pre-filled syringe of claim 11, comprising a coating of flowable lubricant between the plunger and medical barrel.

22. The prefilled syringe of claim 11, wherein the prefilled syringe is a 0.5 mL syringe and the injectable product is a liquid ophthalmic drug formulation.

23. The prefilled syringe of claim 22, wherein the liquid ophthalmic drug formulation is suitable for intravitreal injection and comprises a VEGF antagonist, wherein the VEGF antagonist comprises an anti-VEGF antibody or an antigen-binding fragment of such antibody.

24. The prefilled syringe of claim 22, wherein the liquid ophthalmic drug formulation comprises Ranibizumab and/or Aflibercept.

25. A method of using the prefilled syringe of claim 22, comprising advancing the plunger down the medical barrel to dispense a portion of the liquid ophthalmic drug formulation in a priming step, followed by inserting a needle into a patient's eye tissue wherein the needle provides fluid communication from the product containing area through the dispensing end of the medical barrel and further advancing the plunger down the barrel to inject the ophthalmic drug formulation into the patient's eye tissue.
